(19) United States
(12) Patent Application Publication
(10) Pub. No.: US 2026/0057985 A1
(43) Pub. Date: Feb. 26, 2026

(54) SYSTEM

(71) Applicant: SoftBank Group Corp., Tokyo (JP)

(72) Inventor: Shiho TAKANO, Tokyo (JP)

(21) Appl. No.: 19/297,290

(22) Filed: Aug. 12, 2025

(30) Foreign Application Priority Data
Aug. 23, 2024 (JP) 2024-142599

(51) Int. Cl. G16H 20/00; G06Q 50/22; G06V 40/16; G10L 25/63

(52) U.S. Cl. CPC G16H 20/00; G06Q 50/22; G06V 40/174; G10L 25/63

(57) ABSTRACT

The system according to the embodiment comprises a collection unit, an analysis unit, a provision unit, and a learning unit. The collection unit collects the facial expressions and voice tone of the subject. The analysis unit analyzes the information collected by the collection unit and identifies the emotions of the subject. The provision unit provides the analysis results obtained by the analysis unit in real time. The learning unit performs self-learning to respond to the individual care needs of users based on the information provided by the provision unit.

### CROSS-REFERENCE TO RELATED APPLICATION

The present application claims priority to and incorporates by reference the entire contents of Japanese Patent Application No. 2024-142599 filed in Japan on Aug. 23, 2024.

### BACKGROUND OF THE INVENTION

### Field of the Invention

The technology of this disclosure relates to a system.

### Description of the Related Art

Japanese Patent Application Laid-open No. 2022-180282 discloses a persona chatbot control method executed by at least one processor, comprising: receiving a user utterance, adding the user utterance to a prompt containing instructions related to the character of the chatbot, encoding the prompt, inputting the encoded prompt into a language model, and generating a chatbot utterance in response to the user utterance.

In conventional technology, it is difficult for caregivers and child welfare workers to accurately grasp the emotions of subjects and respond appropriately, and there is room for improvement.

### SUMMARY OF THE INVENTION

The system according to the embodiment comprises a collection unit, an analysis unit, a provision unit, and a learning unit. The collection unit collects the facial expressions and voice tone of the subject. The analysis unit analyzes the information collected by the collection unit and identifies the emotions of the subject. The provision unit provides the analysis results obtained by the analysis unit in real time. The learning unit performs self-learning to respond to the individual care needs of users based on the information provided by the provision unit.

### BRIEF DESCRIPTION OF THE DRAWINGS

FIG. 1 is a conceptual diagram showing an example configuration of a data processing system according to the first embodiment;

FIG. 2 is a conceptual diagram showing an example of main functions of a data processing device and a smart device according to the first embodiment;

FIG. 3 is a conceptual diagram showing an example configuration of a data processing system according to the second embodiment;

FIG. 4 is a conceptual diagram showing an example of main functions of a data processing device and smart glasses according to the second embodiment;

FIG. 5 is a conceptual diagram showing an example configuration of a data processing system according to the third embodiment;

FIG. 6 is a conceptual diagram showing an example of main functions of a data processing device and a headset-type terminal according to the third embodiment;

FIG. 7 is a conceptual diagram showing an example configuration of a data processing system according to the fourth embodiment;

FIG. 8 is a conceptual diagram showing an example of main functions of a data processing device and a robot according to the fourth embodiment;

FIG. 9 shows an emotion map where multiple emotions are mapped; and

FIG. 10 shows an emotion map where multiple emotions are mapped.

### DETAILED DESCRIPTION OF THE PREFERRED EMBODIMENTS

Hereinafter, an example of an embodiment of the system related to the technology disclosed herein will be described with reference to the attached drawings.

First, the terminology used in the following description will be explained.

In the following embodiments, a processor with a sign (hereinafter simply referred to as “processor”) may be a single computing device or a combination of multiple computing devices. The processor may be a single type of computing device or a combination of multiple types of computing devices. Examples of computing devices include a CPU (Central Processing Unit), GPU (Graphics Processing Unit), GPGPU (General-Purpose computing on Graphics Processing Units), APU (Accelerated Processing Unit), or TPU (Tensor Processing Unit), among others.

In the following embodiments, a RAM (Random Access Memory) with a sign is a memory where information is temporarily stored and used as a work memory by the processor.

In the following embodiments, a storage with a sign is one or more non-volatile storage devices for storing various programs and parameters. Examples of non-volatile storage devices include flash memory (SSD (Solid State Drive)), magnetic disks (e.g., hard disks), or magnetic tapes, among others.

In the following embodiments, a communication I/F (Interface) with a sign is an interface including a communication processor and an antenna, among others. The communication I/F manages communication between multiple computers. Examples of communication standards applicable to the communication I/F include wireless communication standards such as 5G (5th Generation Mobile Communication System), Wi-Fi (registered trademark), or Bluetooth (registered trademark), among others.

In the following embodiments, “A and/or B” means “at least one of A and B.” In other words, “A and/or B” means it may be only A, only B, or a combination of A and B. Moreover, when expressing three or more items connected by “and/or,” the same concept as “A and/or B” applies.

### First Embodiment

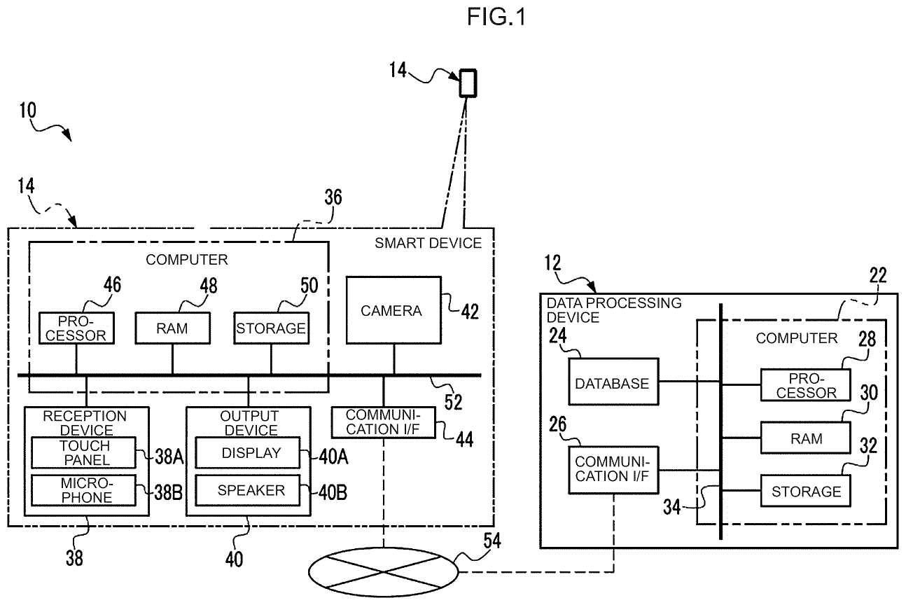

FIG. 1 shows an example configuration of a data processing system 10 according to the first embodiment.

As shown in FIG. 1, the data processing system 10 comprises a data processing device 12 and a smart device 14. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN (Wide Area Network) and/or a LAN (Local Area Network), among others.

The smart device 14 comprises a computer 36, a reception device 38, an output device 40, a camera 42, and a communication I/F 44. The computer 36 comprises a processor 46, RAM 48, and storage 50. The processor 46, RAM 48, and storage 50 are connected to a bus 52. The reception device 38, output device 40, and camera 42 are also connected to the bus 52.

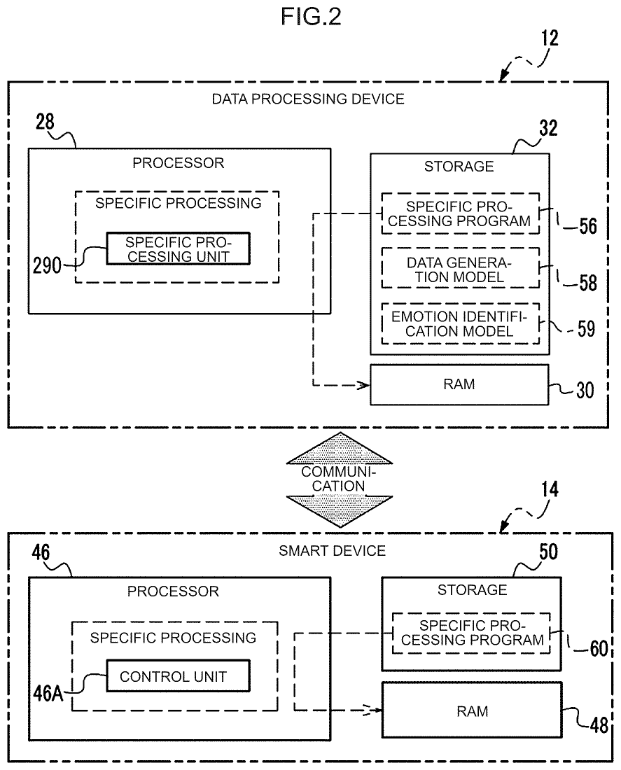

The reception device 38 comprises a touch panel 38A and a microphone 38B, among others, and accepts user input. The touch panel 38A accepts user input by detecting contact from an indicating object (e.g., a pen or finger). The microphone 38B accepts user input by detecting the user's voice. The control unit 46A sends data indicating user input accepted by the touch panel 38A and microphone 38B to the data processing device 12. The data processing device 12 has a specific processing unit 290 (see FIG. 2) that acquires data indicating user input.

The output device 40 comprises a display 40A and a speaker 40B, among others, and presents data to the user by outputting it in a perceptible form (e.g., audio and/or text). The display 40A displays visible information such as text and images according to instructions from the processor 46. The speaker 40B outputs audio according to instructions from the processor 46. The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors.

The communication I/F 44 is connected to the network 54. The communication I/F 44 and 26 manage the exchange of various information between the processor 46 and the processor 28 via the network 54.

FIG. 2 shows an example of the main functions of the data processing device 12 and the smart device 14.

As shown in FIG. 2, specific processing is performed in the data processing device 12 by the processor 28. The storage 32 stores a specific processing program 56. The specific processing program 56 is an example of a “program” related to the technology disclosed herein. The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the smart device 14, specific processing is performed by the processor 46. The storage 50 stores a specific processing program 60. The specific processing program 60 is used in conjunction with the specific processing program 56 by the data processing system 10. The processor 46 reads the specific processing program 60 from the storage 50 and executes it on the RAM 48. The specific processing is realized by the processor 46 operating as a control unit 46A according to the specific processing program 60 executed on the RAM 48. The smart device 14 may also have similar data generation models and emotion identification models as the data generation model 58 and emotion identification model 59, and perform the same processing as the specific processing unit 290 using these models.

Other devices besides the data processing device 12 may have the data generation model 58. For example, a server device (e.g., a generation server) may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.). Next, an example of processing by the data processing system 10 according to the first embodiment will be described.

### Example 1 of the Embodiment

The emotion recognition support system according to the embodiment of the present invention is a system that collects the facial expressions and voice tone of a subject, analyzes these pieces of information using AI to identify emotions, and provides the results in real time. The emotion recognition support system collects the facial expressions and voice tone of the subject, and AI analyzes these pieces of information to identify emotions. The analysis results are provided in real time through an earphone-type device, and appropriate countermeasures are presented. Furthermore, the AI performs self-learning to respond to the individual care needs of each user and presents the optimal approach. For example, the emotion recognition support system collects the facial expressions and voice tone of the subject using a microphone-camera type device. For example, if the subject is speaking with a smile, the system collects that facial expression and voice tone. This information is input to the AI. Next, the emotion recognition support system analyzes the collected information using AI to identify the emotions of the subject. For example, the AI analyzes the smile and bright voice tone and determines that the subject is happy. This analysis result is provided in real time through an earphone-type device. Furthermore, the emotion recognition support system performs self-learning with AI so as to respond to the individual care needs of each user. For example, it learns what kind of emotions a specific subject shows in specific situations and presents the optimal approach based on that information. As a result, the emotion recognition support system can reduce the burden on caregivers and child welfare workers and improve the quality of welfare services. By enabling real-time understanding of the subject's emotions, appropriate responses can be made quickly, thereby reducing the stress of caregivers and child welfare workers. In addition, by responding based on the subject's emotions, the satisfaction of the subject is improved. Furthermore, by having AI perform emotion analysis and present countermeasures, the work of caregivers and child welfare workers is streamlined, and high-quality welfare services can be provided even with a small workforce.

The emotion recognition support system according to the embodiment comprises a collection unit, an analysis unit, a provision unit, and a learning unit. The collection unit collects the facial expressions and voice tone of the subject. The facial expressions of the subject may include, for example, smiles, anger, sadness, etc., but are not limited to such examples. The collection unit collects the facial expressions and voice tone of the subject using, for example, a microphone-camera type device. The collection unit can also collect the voice tone of the subject. For example, the collection unit collects the high pitch, low pitch, and intonation of the subject's voice. The analysis unit analyzes the information collected by the collection unit and identifies the emotions of the subject. The identification of emotions is performed, for example, using an emotion recognition algorithm, but is not limited to such examples. For example, the analysis unit analyzes the collected facial expression data and determines whether the subject is smiling. The analysis unit can also analyze the collected voice tone and determine whether the subject is angry. The analysis unit can also analyze the collected facial expression data and voice tone in combination to comprehensively identify the emotions of the subject. The provision unit provides the analysis results obtained by the analysis unit in real time. The provision may be performed, for example, through an earphone-type device, but is not limited to such examples. For example, the provision unit provides the analysis results to caregivers or child welfare workers through an earphone-type device. The provision unit can also display the analysis results on a display. The provision unit can also notify the analysis results by voice. The learning unit performs self-learning to respond to the individual care needs of users based on the information provided by the provision unit. The self-learning is performed, for example, using a machine learning algorithm, but is not limited to such examples. For example, the learning unit learns what kind of emotions a specific subject shows in specific situations. The learning unit can also learn what kind of responses a specific subject prefers in specific situations. The learning unit can also present the optimal approach based on the learned information. As a result, the emotion recognition support system according to the embodiment can reduce the burden on caregivers and child welfare workers and improve the quality of welfare services.

The emotion recognition support system comprises a collection unit that collects the facial expressions or voice tone of the subject using a microphone-camera type device. The collection unit collects the facial expressions or voice tone of the subject using a microphone-camera type device. The microphone-camera type device may include, for example, a high-resolution camera or a high-sensitivity microphone, but is not limited to such examples. For example, the collection unit collects the facial expressions of the subject in detail using a high-resolution camera. The collection unit can also accurately collect the voice tone of the subject using a high-sensitivity microphone. The collection unit can also collect the natural facial expressions and voice tone of the subject by devising the installation method of the microphone-camera type device. For example, the collection unit installs the camera at the eye level of the subject to collect natural facial expressions. The collection unit can also install the microphone close to the subject's mouth to accurately collect the voice tone. Thus, by using a microphone-camera type device, the facial expressions and voice tone of the subject can be accurately collected.

The analysis unit can analyze the collected information and identify the emotions of the subject. The analysis unit analyzes the collected information and identifies the emotions of the subject. The analysis is performed, for example, using a data analysis algorithm, but is not limited to such examples. For example, the analysis unit analyzes the collected facial expression data and determines whether the subject is smiling. The analysis unit can also analyze the collected voice tone and determine whether the subject is angry. The analysis unit can also analyze the collected facial expression data and voice tone in combination to comprehensively identify the emotions of the subject. For example, the analysis unit analyzes the facial expressions of the subject using a facial recognition algorithm. The analysis unit can also analyze the voice tone of the subject using a voice analysis algorithm. The analysis unit can also use an emotion recognition algorithm to analyze the combination of facial expression data and voice tone to identify the emotions of the subject. Thus, by analyzing the collected information, the emotions of the subject can be accurately identified.

The provision unit can provide the analysis results in real time through an earphone-type device. The provision unit provides the analysis results in real time through an earphone-type device. The earphone-type device may include, for example, high-quality earphones or wireless earphones, but is not limited to such examples. For example, the provision unit provides the analysis results clearly using high-quality earphones. The provision unit can also provide the analysis results using wireless earphones. The provision unit can also provide the analysis results in real time by having caregivers or child welfare workers wear the earphone-type device. For example, the provision unit notifies the analysis results by voice. The provision unit can also notify the analysis results by text message. The provision unit can also display the analysis results on a display. Thus, by providing the analysis results in real time through an earphone-type device, caregivers and child welfare workers can respond quickly.

The learning unit can learn the emotional patterns of a specific subject and present the optimal approach. The learning unit learns the emotional patterns of a specific subject and presents the optimal approach. The learning is performed, for example, using a machine learning algorithm, but is not limited to such examples. For example, the learning unit learns what kind of emotions a specific subject shows in specific situations. The learning unit can also learn what kind of responses a specific subject prefers in specific situations. The learning unit can also present the optimal approach based on the learned information. For example, if the specific subject is feeling stressed, the learning unit presents an approach with a relaxing effect. If the specific subject is relaxed, the learning unit can present an approach that provides detailed information. If the specific subject is excited, the learning unit can present a visually stimulating approach. Thus, by learning the emotional patterns of a specific subject, the optimal approach tailored to individual care needs can be provided.

The provision unit can aim to reduce the burden on caregivers and child welfare workers and improve the quality of welfare services. The provision unit aims to reduce the burden on caregivers and child welfare workers and improve the quality of welfare services. The burden reduction is performed, for example, by shortening work time or reducing stress, but is not limited to such examples. For example, the provision unit shortens the work time of caregivers and child welfare workers by providing emotion analysis results in real time. The provision unit can also reduce the stress of caregivers and child welfare workers by presenting appropriate countermeasures. The provision unit also aims to improve the quality of welfare services. The quality improvement is performed, for example, through service evaluation criteria or improvement methods, but is not limited to such examples. For example, the provision unit sets service evaluation criteria based on emotion analysis results. The provision unit can also propose service improvement methods based on emotion analysis results. Thus, the burden on caregivers and child welfare workers can be reduced and the quality of welfare services can be improved.

The emotion recognition support system comprises a collection unit that optimizes the collection method by referring to the subject's past emotional data at the time of collection. The collection unit optimizes the collection method by referring to the subject's past emotional data at the time of collection. The optimization is performed, for example, by adjusting algorithms or setting parameters, but is not limited to such examples. For example, the collection unit refers to situations in which the subject felt stressed in the past and focuses on collecting facial expressions and voice tone in similar situations. The collection unit can also refer to situations in which the subject was relaxed in the past and adjust the collection method based on the data from that time. The collection unit can also refer to situations in which the subject was excited in the past and optimize the collection method based on the data from that time. Thus, by referring to past emotional data, the collection method can be optimized and more accurate data collection becomes possible. Some or all of the above-described processing in the collection unit may be performed using AI, or may be performed without using AI. For example, the collection unit can input past emotional data into an AI model, and the AI can propose the optimal collection method.

The emotion recognition support system comprises a collection unit that adjusts the collection means based on the subject's current environment at the time of collection. The collection unit adjusts the collection means based on the subject's current environment (indoors/outdoors, noise level, etc.) at the time of collection. The adjustment is performed, for example, by selecting the collection means or changing settings, but is not limited to such examples. For example, if the environment is quiet indoors, the collection unit collects subtle changes in voice tone. If the environment is noisy outdoors, the collection unit can prioritize collecting changes in facial expressions. If the noise level is moderate, the collection unit can collect both facial expressions and voice tone in a balanced manner. Thus, by adjusting the collection means based on the current environment, more appropriate data collection becomes possible. Some or all of the above-described processing in the collection unit may be performed using AI, or may be performed without using AI. For example, the collection unit can input current environment data into an AI model, and the AI can propose the optimal collection means.

The emotion recognition support system comprises a collection unit that corrects the collected data by considering the subject's physical condition at the time of collection. The collection unit corrects the collected data by considering the subject's physical condition (fatigue, health status, etc.) at the time of collection. The correction is performed, for example, by adjusting or filtering the data, but is not limited to such examples. For example, if the subject is tired, the collection unit focuses on collecting changes in facial expressions and corrects changes in voice tone. If the subject is in good health, the collection unit can collect both facial expressions and voice tone in a balanced manner. If the subject is in poor physical condition, the collection unit can focus on collecting changes in voice tone and correct changes in facial expressions. Thus, by correcting the collected data considering the physical condition, more accurate data collection becomes possible. Some or all of the above-described processing in the collection unit may be performed using AI, or may be performed without using AI. For example, the collection unit can input physical condition data into an AI model, and the AI can perform the correction of the collected data.

The emotion recognition support system comprises a collection unit that enhances the relevance of collected data by considering the subject's geographic location information at the time of collection. The collection unit enhances the relevance of collected data by considering the subject's geographic location information at the time of collection. The enhancement of relevance is performed, for example, by selecting or filtering data, but is not limited to such examples. For example, if the subject is in a park, the collection unit removes natural sounds and collects facial expressions and voice tone. If the subject is in an office, the collection unit can remove background conversation sounds and collect facial expressions and voice tone. If the subject is at home, the collection unit can remove household noise and collect facial expressions and voice tone. Thus, by considering geographic location information, the relevance of collected data can be enhanced and more accurate data collection becomes possible. Some or all of the above-described processing in the collection unit may be performed using AI, or may be performed without using AI. For example, the collection unit can input geographic location information into an AI model, and the AI can perform filtering to enhance the relevance of collected data.

The emotion recognition support system comprises a collection unit that analyzes the subject's social media activity at the time of collection and supplements relevant emotional data. The collection unit analyzes the subject's social media activity at the time of collection and supplements relevant emotional data. The supplementation is performed, for example, by adding or correcting data, but is not limited to such examples. For example, the collection unit analyzes the content of the subject's social media posts and supplements changes in emotions. The collection unit can also refer to the activities of the subject's friends on social media to supplement emotional data. The collection unit can also supplement emotional data based on the subject's check-in information on social media. Thus, by analyzing social media activity, relevant emotional data can be supplemented and more accurate emotion analysis becomes possible. Some or all of the above-described processing in the collection unit may be performed using AI, or may be performed without using AI. For example, the collection unit can input social media activity data into an AI model, and the AI can perform supplementation of emotional data.

The emotion recognition support system comprises a collection unit that customizes the collection method by reflecting the subject's past feedback at the time of collection. The collection unit customizes the collection method by reflecting the subject's past feedback at the time of collection. The customization is performed, for example, by selecting the collection means or changing settings, but is not limited to such examples. For example, the collection unit adjusts the collection method based on feedback previously provided by the subject. The collection unit can also preferentially use collection methods that the subject has preferred in the past. The collection unit can also analyze the subject's past feedback and propose the optimal collection method. Thus, by reflecting past feedback, the collection method can be customized and more accurate data collection becomes possible. Some or all of the above-described processing in the collection unit may be performed using AI, or may be performed without using AI. For example, the collection unit can input past feedback data into an AI model, and the AI can perform customization of the collection method.

The emotion recognition support system comprises an analysis unit that improves accuracy by comparing the subject's emotional patterns with past data during analysis. The analysis unit improves accuracy by comparing the subject's emotional patterns with past data during analysis. The improvement of accuracy is performed, for example, by improving data accuracy or algorithms, but is not limited to such examples. For example, the analysis unit analyzes by comparing the current emotional patterns with the subject's past emotional data. The analysis unit can also refer to the subject's past emotional patterns to more accurately identify current emotions. The analysis unit can also use the subject's past emotional data to improve the accuracy of the analysis algorithm. Thus, by comparing with past data, the accuracy of emotion analysis can be improved. Some or all of the above-described processing in the analysis unit may be performed using AI, or may be performed without using AI. For example, the analysis unit can input past emotional data into an AI model, and the AI can perform analysis for accuracy improvement.

The emotion recognition support system comprises an analysis unit that tracks fluctuations in the subject's emotions in real time during analysis and updates the analysis results. The analysis unit tracks fluctuations in the subject's emotions in real time during analysis and updates the analysis results. The tracking is performed, for example, by monitoring emotional fluctuations in real time and updating the analysis results each time a fluctuation occurs, but is not limited to such examples. For example, the analysis unit updates the analysis results in real time each time the subject's emotions fluctuate. The analysis unit can also track fluctuations in the subject's emotions in real time and provide the latest analysis results. The analysis unit can also immediately update the analysis results if the subject's emotions fluctuate rapidly. Thus, by tracking emotional fluctuations in real time, the latest analysis results can be provided. Some or all of the above-described processing in the analysis unit may be performed using AI, or may be performed without using AI. For example, the analysis unit can input real-time emotional data into an AI model, and the AI can perform updating of the analysis results.

The emotion recognition support system comprises an analysis unit that considers external factors affecting the subject's emotions during analysis. The analysis unit considers external factors (such as weather, time of day, etc.) affecting the subject's emotions during analysis. The consideration is performed, for example, by collecting data on external factors and reflecting them in emotion analysis, but is not limited to such examples. For example, the analysis unit analyzes by considering the impact of bad weather on the subject's emotions. The analysis unit can also analyze by considering emotional fluctuations due to time of day. The analysis unit can also analyze by considering external factors (such as noise level, influence of surrounding people, etc.). Thus, by considering external factors, more accurate emotion analysis becomes possible. Some or all of the above-described processing in the analysis unit may be performed using AI, or may be performed without using AI. For example, the analysis unit can input external factor data into an AI model and reflect it in emotion analysis.

The emotion recognition support system comprises an analysis unit that performs emotion analysis by considering the subject's geographic background during analysis. The analysis unit performs emotion analysis by considering the subject's geographic background during analysis. The consideration is performed, for example, by collecting data on geographic background and reflecting it in emotion analysis, but is not limited to such examples. For example, if the subject is in an urban area, the analysis unit analyzes by considering urban-specific stress factors. If the subject is in a natural environment, the analysis unit can analyze by considering relaxation effects. If the subject is in a specific region, the analysis unit can analyze by considering the culture and customs specific to that region. Thus, by considering geographic background, more accurate emotion analysis becomes possible. Some or all of the above-described processing in the analysis unit may be performed using AI, or may be performed without using AI. For example, the analysis unit can input geographic background data into an AI model and reflect it in emotion analysis.

The emotion recognition support system comprises an analysis unit that improves analysis accuracy by referring to relevant literature and research data on the subject during analysis. The analysis unit improves analysis accuracy by referring to relevant literature and research data on the subject during analysis. The reference is performed, for example, by collecting and analyzing relevant literature and research data, but is not limited to such examples. For example, the analysis unit analyzes by referring to the latest research data on the subject's emotions. The analysis unit can also adjust the analysis algorithm based on relevant literature on the subject's emotions. The analysis unit can also improve analysis accuracy by referring to past research data on the subject's emotions. Thus, by referring to relevant literature and research data, analysis accuracy can be improved. Some or all of the above-described processing in the analysis unit may be performed using AI, or may be performed without using AI. For example, the analysis unit can input relevant literature and research data into an AI model, and the AI can perform analysis accuracy improvement.

The emotion recognition support system comprises an analysis unit that analyzes emotions by considering the subject's social background during analysis. The analysis unit analyzes emotions by considering the subject's social background (such as culture, customs, etc.) during analysis. The consideration is performed, for example, by collecting data on social background and reflecting it in emotion analysis, but is not limited to such examples. For example, the analysis unit analyzes emotions by considering the subject's cultural background. The analysis unit can also analyze emotions by considering the subject's customs and lifestyle. The analysis unit can also analyze emotional fluctuations based on the subject's social background. Thus, by considering social background, more accurate emotion analysis becomes possible. Some or all of the above-described processing in the analysis unit may be performed using AI, or may be performed without using AI. For example, the analysis unit can input social background data into an AI model and reflect it in emotion analysis.

The emotion recognition support system comprises a provision unit that optimizes the information provision method by referring to the subject's past responses at the time of provision. The provision unit optimizes the information provision method by referring to the subject's past responses at the time of provision. The optimization is performed, for example, by selecting the information provision means or changing settings, but is not limited to such examples. For example, the provision unit preferentially uses information provision methods that the subject has preferred in the past. The provision unit can also propose the optimal information provision method based on the subject's past responses. The provision unit can also analyze the subject's past responses and optimize the information provision method. Thus, by referring to past responses, the information provision method can be optimized and more appropriate information provision becomes possible. Some or all of the above-described processing in the provision unit may be performed using AI, or may be performed without using AI. For example, the provision unit can input past response data into an AI model, and the AI can perform optimization of the information provision method.

The emotion recognition support system comprises a provision unit that adjusts information provision based on the subject's current situation at the time of provision. The provision unit adjusts information provision based on the subject's current situation (such as being active or on break) at the time of provision. The adjustment is performed, for example, by changing the timing or format of information provision, but is not limited to such examples. For example, if the subject is active, the provision unit provides concise and to-the-point information. If the subject is on break, the provision unit can provide detailed information. The provision unit can also propose the optimal information provision method based on the subject's current situation. Thus, by adjusting information provision based on the current situation, more appropriate information provision becomes possible. Some or all of the above-described processing in the provision unit may be performed using AI, or may be performed without using AI. For example, the provision unit can input current situation data into an AI model, and the AI can perform adjustment of information provision.

The emotion recognition support system comprises a provision unit that corrects information provision by considering the subject's physical condition at the time of provision. The provision unit corrects information provision by considering the subject's physical condition (such as fatigue, health status, etc.) at the time of provision. The correction is performed, for example, by changing the content or format of information, but is not limited to such examples. For example, if the subject is tired, the provision unit provides concise and to-the-point information. If the subject is in good health, the provision unit can provide detailed information. If the subject is in poor physical condition, the provision unit can provide information with a relaxing effect. Thus, by correcting information provision considering the physical condition, more appropriate information provision becomes possible. Some or all of the above-described processing in the provision unit may be performed using AI, or may be performed without using AI. For example, the provision unit can input physical condition data into an AI model, and the AI can perform correction of information provision.

The emotion recognition support system comprises a provision unit that provides highly relevant information by considering the subject's geographic location information at the time of provision. The provision unit provides highly relevant information by considering the subject's geographic location information at the time of provision. The enhancement of relevance is performed, for example, by selecting or filtering information, but is not limited to such examples. For example, if the subject is in a specific region, the provision unit provides information related to that region. If the subject is traveling, the provision unit can provide information related to the travel destination. If the subject is at home, the provision unit can provide information about the area around the home. Thus, by considering geographic location information, highly relevant information provision becomes possible. Some or all of the above-described processing in the provision unit may be performed using AI, or may be performed without using AI. For example, the provision unit can input geographic location information into an AI model, and the AI can select highly relevant information.

The emotion recognition support system comprises a provision unit that analyzes the subject's social media activity at the time of provision and provides relevant information. The provision unit analyzes the subject's social media activity at the time of provision and provides relevant information. The analysis is performed, for example, by analyzing the content and reactions of social media posts, but is not limited to such examples. For example, the provision unit provides information about places where the subject has checked in on social media. The provision unit can also analyze the content of the subject's social media posts and provide relevant information. The provision unit can also refer to the activities of the subject's friends on social media to provide relevant information. Thus, by analyzing social media activity, relevant information provision becomes possible. Some or all of the above-described processing in the provision unit may be performed using AI, or may be performed without using AI. For example, the provision unit can input social media activity data into an AI model, and the AI can select relevant information.

The emotion recognition support system comprises a provision unit that customizes the information provision method by reflecting the subject's past feedback at the time of provision. The provision unit customizes the information provision method by reflecting the subject's past feedback at the time of provision. The customization is performed, for example, by selecting the information provision means or changing settings, but is not limited to such examples. For example, the provision unit preferentially uses information provision methods that the subject has preferred in the past. The provision unit can also propose the optimal information provision method based on the subject's past feedback. The provision unit can also analyze the subject's past feedback and customize the information provision method. Thus, by reflecting past feedback, the information provision method can be customized and more appropriate information provision becomes possible. Some or all of the above-described processing in the provision unit may be performed using AI, or may be performed without using AI. For example, the provision unit can input past feedback data into an AI model, and the AI can perform customization of the information provision method.

The emotion recognition support system comprises a learning unit that optimizes the learning algorithm by referring to past learning data during learning. The learning unit optimizes the learning algorithm by referring to past learning data during learning. The optimization is performed, for example, by adjusting algorithms or setting parameters, but is not limited to such examples. For example, the learning unit adjusts the current learning algorithm based on past learning data. The learning unit can also refer to past learning data to find the optimal learning pattern. The learning unit can also use past learning data to improve the accuracy of the learning algorithm. Thus, by referring to past learning data, the learning algorithm can be optimized and the accuracy of learning can be improved. Some or all of the above-described processing in the learning unit may be performed using AI, or may be performed without using AI. For example, the learning unit can input past learning data into an AI model, and the AI can perform optimization of the learning algorithm.

The emotion recognition support system comprises a learning unit that analyzes fluctuations in the subject's emotional patterns during learning and adjusts the update frequency of learning data. The learning unit analyzes fluctuations in the subject's emotional patterns during learning and adjusts the update frequency of learning data. The adjustment is performed, for example, by setting the update frequency or weighting data, but is not limited to such examples. For example, if the subject's emotional patterns fluctuate frequently, the learning unit increases the update frequency of learning data. If the subject's emotional patterns are stable, the learning unit can decrease the update frequency of learning data. The learning unit can also analyze fluctuations in the subject's emotional patterns and set the optimal update frequency. Thus, by analyzing fluctuations in emotional patterns, the update frequency of learning data can be optimized and the accuracy of learning can be improved. Some or all of the above-described processing in the learning unit may be performed using AI, or may be performed without using AI. For example, the learning unit can input fluctuation data of emotional patterns into an AI model, and the AI can perform adjustment of the update frequency.

The emotion recognition support system comprises a learning unit that updates learning data by reflecting the subject's feedback during learning. The learning unit updates learning data by reflecting the subject's feedback during learning. The update is performed, for example, by collecting and analyzing feedback, but is not limited to such examples. For example, the learning unit updates learning data based on feedback provided by the subject. The learning unit can also analyze the subject's feedback and adjust the learning algorithm. The learning unit can also improve the accuracy of learning data by reflecting the subject's feedback. Thus, by reflecting feedback, learning data can be updated and the accuracy of learning can be improved. Some or all of the above-described processing in the learning unit may be performed using AI, or may be performed without using AI. For example, the learning unit can input feedback data into an AI model, and the AI can perform updating of learning data.

The emotion recognition support system comprises a learning unit that weights learning data based on the submission timing of the subject's emotional data during learning. The learning unit weights learning data based on the submission timing of the subject's emotional data during learning. The weighting is performed, for example, based on the importance or freshness of the data, but is not limited to such examples. For example, the learning unit weights learning data based on the timing when the subject submitted emotional data. The learning unit can also adjust the learning algorithm by considering the submission timing of the subject's emotional data. The learning unit can also set the priority of learning data based on the submission timing of the subject's emotional data. Thus, by weighting learning data based on the submission timing of emotional data, the accuracy of learning can be improved. Some or all of the above-described processing in the learning unit may be performed using AI, or may be performed without using AI. For example, the learning unit can input the submission timing of emotional data into an AI model, and the AI can perform

The emotion recognition support system comprises a learning unit that expands learning data by integrating information from different data sources during learning. The learning unit expands learning data by integrating information from different data sources during learning. The integration is performed, for example, by collecting and integrating data, but is not limited to such examples. For example, the learning unit integrates emotional data from different data sources for learning. The learning unit can also expand learning data based on information from different data sources. The learning unit can also use data from different data sources to improve the accuracy of the learning algorithm. Thus, by integrating information from different data sources, learning data can be expanded and the accuracy of learning can be improved. Some or all of the above-described processing in the learning unit may be performed using AI, or may be performed without using AI. For example, the learning unit can input information from different data sources into an AI model, and the AI can perform data integration.

The emotion recognition support system comprises a learning unit that adjusts the learning algorithm by considering the subject's social background during learning. The learning unit adjusts the learning algorithm by considering the subject's social background (such as culture, customs, etc.) during learning. The adjustment is performed, for example, by setting algorithm parameters or weighting, but is not limited to such examples. For example, the learning unit adjusts the learning algorithm by considering the subject's cultural background. The learning unit can also adjust the learning algorithm by considering the subject's customs and lifestyle. The learning unit can also optimize the learning algorithm based on the subject's social background. Thus, by considering social background, the learning algorithm can be optimized and the accuracy of learning can be improved. Some or all of the above-described processing in the learning unit may be performed using AI, or may be performed without using AI. For example, the learning unit can input social background data into an AI model, and the AI can perform adjustment of the algorithm.

The system according to the embodiment is not limited to the examples described above, and various modifications are possible, for example, as follows.

The emotion recognition support system comprises a collection unit that optimizes the collection method by referring to the subject's past emotional data at the time of collection. The collection unit optimizes the collection method by referring to the subject's past emotional data at the time of collection. The optimization is performed, for example, by adjusting algorithms or setting parameters, but is not limited to such examples. For example, the collection unit refers to situations in which the subject felt stressed in the past and focuses on collecting facial expressions and voice tone in similar situations. The collection unit can also refer to situations in which the subject was relaxed in the past and adjust the collection method based on the data from that time. The collection unit can also refer to situations in which the subject was excited in the past and optimize the collection method based on the data from that time. Thus, by referring to past emotional data, the collection method can be optimized and more accurate data collection becomes possible. Some or all of the above-described processing in the collection unit may be performed using AI, or may be performed without using AI. For example, the collection unit can input past emotional data into an AI model, and the AI can propose the optimal collection method.

The emotion recognition support system comprises a collection unit that adjusts the collection means based on the subject's current environment at the time of collection. The collection unit adjusts the collection means based on the subject's current environment (indoors/outdoors, noise level, etc.) at the time of collection. The adjustment is performed, for example, by selecting the collection means or changing settings, but is not limited to such examples. For example, if the environment is quiet indoors, the collection unit collects subtle changes in voice tone. If the environment is noisy outdoors, the collection unit can prioritize collecting changes in facial expressions. If the noise level is moderate, the collection unit can collect both facial expressions and voice tone in a balanced manner. Thus, by adjusting the collection means based on the current environment, more appropriate data collection becomes possible. Some or all of the above-described processing in the collection unit may be performed using AI, or may be performed without using AI. For example, the collection unit can input current environment data into an AI model, and the AI can propose the optimal collection means.

The emotion recognition support system comprises a collection unit that corrects the collected data by considering the subject's physical condition at the time of collection. The collection unit corrects the collected data by considering the subject's physical condition (fatigue, health status, etc.) at the time of collection. The correction is performed, for example, by adjusting or filtering the data, but is not limited to such examples. For example, if the subject is tired, the collection unit focuses on collecting changes in facial expressions and corrects changes in voice tone. If the subject is in good health, the collection unit can collect both facial expressions and voice tone in a balanced manner. If the subject is in poor physical condition, the collection unit can focus on collecting changes in voice tone and correct changes in facial expressions. Thus, by correcting the collected data considering the physical condition, more accurate data collection becomes possible. Some or all of the above-described processing in the collection unit may be performed using AI, or may be performed without using AI. For example, the collection unit can input physical condition data into an AI model, and the AI can perform the correction of the collected data.

The emotion recognition support system comprises a collection unit that enhances the relevance of collected data by considering the subject's geographic location information at the time of collection. The collection unit enhances the relevance of collected data by considering the subject's geographic location information at the time of collection. The enhancement of relevance is performed, for example, by selecting or filtering data, but is not limited to such examples. For example, if the subject is in a park, the collection unit removes natural sounds and collects facial expressions and voice tone. If the subject is in an office, the collection unit can remove background conversation sounds and collect facial expressions and voice tone. If the subject is at home, the collection unit can remove household noise and collect facial expressions and voice tone. Thus, by considering geographic location information, the relevance of collected data can be enhanced and more accurate data collection becomes possible. Some or all of the above-described processing in the collection unit may be performed using AI, or may be performed without using AI. For example, the collection unit can input geographic location information into an AI model, and the AI can perform filtering to enhance the relevance of collected data.

The emotion recognition support system comprises a collection unit that analyzes the subject's social media activity at the time of collection and supplements relevant emotional data. The collection unit analyzes the subject's social media activity at the time of collection and supplements relevant emotional data. The supplementation is performed, for example, by adding or correcting data, but is not limited to such examples. For example, the collection unit analyzes the content of the subject's social media posts and supplements changes in emotions. The collection unit can also refer to the activities of the subject's friends on social media to supplement emotional data. The collection unit can also supplement emotional data based on the subject's check-in information on social media. Thus, by analyzing social media activity, relevant emotional data can be supplemented and more accurate emotion analysis becomes possible. Some or all of the above-described processing in the collection unit may be performed using AI, or may be performed without using AI. For example, the collection unit can input social media activity data into an AI model, and the AI can perform supplementation of emotional data.

The emotion recognition support system comprises a collection unit that customizes the collection method by reflecting the subject's past feedback at the time of collection. The collection unit customizes the collection method by reflecting the subject's past feedback at the time of collection. The customization is performed, for example, by selecting the collection means or changing settings, but is not limited to such examples. For example, the collection unit adjusts the collection method based on feedback previously provided by the subject. The collection unit can also preferentially use collection methods that the subject has preferred in the past. The collection unit can also analyze the subject's past feedback and propose the optimal collection method. Thus, by reflecting past feedback, the collection method can be customized and more accurate data collection becomes possible. Some or all of the above-described processing in the collection unit may be performed using AI, or may be performed without using AI. For example, the collection unit can input past feedback data into an AI model, and the AI can perform customization of the collection method.

The emotion recognition support system comprises an analysis unit that improves accuracy by comparing the subject's emotional patterns with past data during analysis. The analysis unit improves accuracy by comparing the subject's emotional patterns with past data during analysis. The improvement of accuracy is performed, for example, by improving data accuracy or algorithms, but is not limited to such examples. For example, the analysis unit analyzes by comparing the current emotional patterns with the subject's past emotional data. The analysis unit can also refer to the subject's past emotional patterns to more accurately identify current emotions. The analysis unit can also use the subject's past emotional data to improve the accuracy of the analysis algorithm. Thus, by comparing with past data, the accuracy of emotion analysis can be improved. Some or all of the above-described processing in the analysis unit may be performed using AI, or may be performed without using AI. For example, the analysis unit can input past emotional data into an AI model, and the AI can perform analysis for accuracy improvement.

The emotion recognition support system comprises an analysis unit that tracks fluctuations in the subject's emotions in real time during analysis and updates the analysis results. The analysis unit tracks fluctuations in the subject's emotions in real time during analysis and updates the analysis results. The tracking is performed, for example, by monitoring emotional fluctuations in real time and updating the analysis results each time a fluctuation occurs, but is not limited to such examples. For example, the analysis unit updates the analysis results in real time each time the subject's emotions fluctuate. The analysis unit can also track fluctuations in the subject's emotions in real time and provide the latest analysis results. The analysis unit can also immediately update the analysis results if the subject's emotions fluctuate rapidly. Thus, by tracking emotional fluctuations in real time, the latest analysis results can be provided. Some or all of the above-described processing in the analysis unit may be performed using AI, or may be performed without using AI. For example, the analysis unit can input real-time emotional data into an AI model, and the AI can perform updating of the analysis results.

The emotion recognition support system comprises an analysis unit that considers external factors affecting the subject's emotions during analysis. The analysis unit considers external factors (such as weather, time of day, etc.) affecting the subject's emotions during analysis. The consideration is performed, for example, by collecting data on external factors and reflecting them in emotion analysis, but is not limited to such examples. For example, the analysis unit analyzes by considering the impact of bad weather on the subject's emotions. The analysis unit can also analyze by considering emotional fluctuations due to time of day. The analysis unit can also analyze by considering external factors (such as noise level, influence of surrounding people, etc.). Thus, by considering external factors, more accurate emotion analysis becomes possible. Some or all of the above-described processing in the analysis unit may be performed using AI, or may be performed without using AI. For example, the analysis unit can input external factor data into an AI model and reflect it in emotion analysis.

The emotion recognition support system comprises an analysis unit that performs emotion analysis by considering the subject's geographic background during analysis. The analysis unit performs emotion analysis by considering the subject's geographic background during analysis. The consideration is performed, for example, by collecting data on geographic background and reflecting it in emotion analysis, but is not limited to such examples. For example, if the subject is in an urban area, the analysis unit analyzes by considering urban-specific stress factors. If the subject is in a natural environment, the analysis unit can analyze by considering relaxation effects. If the subject is in a specific region, the analysis unit can analyze by considering the culture and customs specific to that region. Thus, by considering geographic background, more accurate emotion analysis becomes possible. Some or all of the above-described processing in the analysis unit may be performed using AI, or may be performed without using AI. For example, the analysis unit can input geographic background data into an AI model and reflect it in emotion analysis.

The emotion recognition support system comprises an analysis unit that improves analysis accuracy by referring to relevant literature and research data on the subject during analysis. The analysis unit improves analysis accuracy by referring to relevant literature and research data on the subject during analysis. The reference is performed, for example, by collecting and analyzing relevant literature and research data, but is not limited to such examples. For example, the analysis unit analyzes by referring to the latest research data on the subject's emotions. The analysis unit can also adjust the analysis algorithm based on relevant literature on the subject's emotions. The analysis unit can also improve analysis accuracy by referring to past research data on the subject's emotions. Thus, by referring to relevant literature and research data, analysis accuracy can be improved. Some or all of the above-described processing in the analysis unit may be performed using AI, or may be performed without using AI. For example, the analysis unit can input relevant literature and research data into an AI model, and the AI can perform analysis accuracy improvement.

The emotion recognition support system comprises an analysis unit that analyzes emotions by considering the subject's social background during analysis. The analysis unit analyzes emotions by considering the subject's social background (such as culture, customs, etc.) during analysis. The consideration is performed, for example, by collecting data on social background and reflecting it in emotion analysis, but is not limited to such examples. For example, the analysis unit analyzes emotions by considering the subject's cultural background. The analysis unit can also analyze emotions by considering the subject's customs and lifestyle. The analysis unit can also analyze emotional fluctuations based on the subject's social background. Thus, by considering social background, more accurate emotion analysis becomes possible. Some or all of the above-described processing in the analysis unit may be performed using AI, or may be performed without using AI. For example, the analysis unit can input social background data into an AI model and reflect it in emotion analysis.

The emotion recognition support system comprises a provision unit that optimizes the information provision method by referring to the subject's past responses at the time of provision. The provision unit optimizes the information provision method by referring to the subject's past responses at the time of provision. The optimization is performed, for example, by selecting the information provision means or changing settings, but is not limited to such examples. For example, the provision unit preferentially uses information provision methods that the subject has preferred in the past. The provision unit can also propose the optimal information provision method based on the subject's past responses. The provision unit can also analyze the subject's past responses and optimize the information provision method. Thus, by referring to past responses, the information provision method can be optimized and more appropriate information provision becomes possible. Some or all of the above-described processing in the provision unit may be performed using AI, or may be performed without using AI. For example, the provision unit can input past response data into an AI model, and the AI can perform optimization of the information provision method.

The emotion recognition support system comprises a provision unit that adjusts information provision based on the subject's current situation at the time of provision. The provision unit adjusts information provision based on the subject's current situation (such as being active or on break) at the time of provision. The adjustment is performed, for example, by changing the timing or format of information provision, but is not limited to such examples. For example, if the subject is active, the provision unit provides concise and to-the-point information. If the subject is on break, the provision unit can provide detailed information. The provision unit can also propose the optimal information provision method based on the subject's current situation. Thus, by adjusting information provision based on the current situation, more appropriate information provision becomes possible. Some or all of the above-described processing in the provision unit may be performed using AI, or may be performed without using AI. For example, the provision unit can input current situation data into an AI model, and the AI can perform adjustment of information provision.

The emotion recognition support system comprises a provision unit that corrects information provision by considering the subject's physical condition at the time of provision. The provision unit corrects information provision by considering the subject's physical condition (such as fatigue, health status, etc.) at the time of provision. The correction is performed, for example, by changing the content or format of information, but is not limited to such examples. For example, if the subject is tired, the provision unit provides concise and to-the-point information. If the subject is in good health, the provision unit can provide detailed information. If the subject is in poor physical condition, the provision unit can provide information with a relaxing effect. Thus, by correcting information provision considering the physical condition, more appropriate information provision becomes possible. Some or all of the above-described processing in the provision unit may be performed using AI, or may be performed without using AI. For example, the provision unit can input physical condition data into an AI model, and the AI can perform correction of information provision.

The emotion recognition support system comprises a provision unit that provides highly relevant information by considering the subject's geographic location information at the time of provision. The provision unit provides highly relevant information by considering the subject's geographic location information at the time of provision. The enhancement of relevance is performed, for example, by selecting or filtering information, but is not limited to such examples. For example, if the subject is in a specific region, the provision unit provides information related to that region. If the subject is traveling, the provision unit can provide information related to the travel destination. If the subject is at home, the provision unit can provide information about the area around the home. Thus, by considering geographic location information, highly relevant information provision becomes possible. Some or all of the above-described processing in the provision unit may be performed using AI, or may be performed without using AI. For example, the provision unit can input geographic location information into an AI model, and the AI can select highly relevant information.

The emotion recognition support system comprises a provision unit that analyzes the subject's social media activity at the time of provision and provides relevant information. The provision unit analyzes the subject's social media activity at the time of provision and provides relevant information. The analysis is performed, for example, by analyzing the content and reactions of social media posts, but is not limited to such examples. For example, the provision unit provides information about places where the subject has checked in on social media. The provision unit can also analyze the content of the subject's social media posts and provide relevant information. The provision unit can also refer to the activities of the subject's friends on social media to provide relevant information. Thus, by analyzing social media activity, relevant information provision becomes possible. Some or all of the above-described processing in the provision unit may be performed using AI, or may be performed without using AI. For example, the provision unit can input social media activity data into an AI model, and the AI can select relevant information.

The emotion recognition support system comprises a provision unit that customizes the information provision method by reflecting the subject's past feedback at the time of provision. The provision unit customizes the information provision method by reflecting the subject's past feedback at the time of provision. The customization is performed, for example, by selecting the information provision means or changing settings, but is not limited to such examples. For example, the provision unit preferentially uses information provision methods that the subject has preferred in the past. The provision unit can also propose the optimal information provision method based on the subject's past feedback. The provision unit can also analyze the subject's past feedback and customize the information provision method. Thus, by reflecting past feedback, the information provision method can be customized and more appropriate information provision becomes possible. Some or all of the above-described processing in the provision unit may be performed using AI, or may be performed without using AI. For example, the provision unit can input past feedback data into an AI model, and the AI can perform customization of the information provision method.

The emotion recognition support system comprises a learning unit that optimizes the learning algorithm by referring to past learning data during learning. The learning unit optimizes the learning algorithm by referring to past learning data during learning. The optimization is performed, for example, by adjusting algorithms or setting parameters, but is not limited to such examples. For example, the learning unit adjusts the current learning algorithm based on past learning data. The learning unit can also refer to past learning data to find the optimal learning pattern. The learning unit can also use past learning data to improve the accuracy of the learning algorithm. Thus, by referring to past learning data, the learning algorithm can be optimized and the accuracy of learning can be improved. Some or all of the above-described processing in the learning unit may be performed using AI, or may be performed without using AI. For example, the learning unit can input past learning data into an AI model, and the AI can perform optimization of the learning algorithm.

The emotion recognition support system comprises a learning unit that analyzes fluctuations in the subject's emotional patterns during learning and adjusts the update frequency of learning data. The learning unit analyzes fluctuations in the subject's emotional patterns during learning and adjusts the update frequency of learning data. The adjustment is performed, for example, by setting the update frequency or weighting data, but is not limited to such examples. For example, if the subject's emotional patterns fluctuate frequently, the learning unit increases the update frequency of learning data. If the subject's emotional patterns are stable, the learning unit can decrease the update frequency of learning data. The learning unit can also analyze fluctuations in the subject's emotional patterns and set the optimal update frequency. Thus, by analyzing fluctuations in emotional patterns, the update frequency of learning data can be optimized and the accuracy of learning can be improved. Some or all of the above-described processing in the learning unit may be performed using AI, or may be performed without using AI. For example, the learning unit can input fluctuation data of emotional patterns into an AI model, and the AI can perform adjustment of the update frequency.

The emotion recognition support system comprises a learning unit that updates learning data by reflecting the subject's feedback during learning. The learning unit updates learning data by reflecting the subject's feedback during learning. The update is performed, for example, by collecting and analyzing feedback, but is not limited to such examples. For example, the learning unit updates learning data based on feedback provided by the subject. The learning unit can also analyze the subject's feedback and adjust the learning algorithm. The learning unit can also improve the accuracy of learning data by reflecting the subject's feedback. Thus, by reflecting feedback, learning data can be updated and the accuracy of learning can be improved. Some or all of the above-described processing in the learning unit may be performed using AI, or may be performed without using AI. For example, the learning unit can input feedback data into an AI model, and the AI can perform updating of learning data.

The emotion recognition support system comprises a learning unit that weights learning data based on the submission timing of the subject's emotional data during learning. The learning unit weights learning data based on the submission timing of the subject's emotional data during learning. The weighting is performed, for example, based on the importance or freshness of the data, but is not limited to such examples. For example, the learning unit weights learning data based on the timing when the subject submitted emotional data. The learning unit can also adjust the learning algorithm by considering the submission timing of the subject's emotional data. The learning unit can also set the priority of learning data based on the submission timing of the subject's emotional data. Thus, by weighting learning data based on the submission timing of emotional data, the accuracy of learning can be improved. Some or all of the above-described processing in the learning unit may be performed using AI, or may be performed without using AI. For example, the learning unit can input the submission timing of emotional data into an AI model, and the AI can perform weighting.

The emotion recognition support system comprises a learning unit that expands learning data by integrating information from different data sources during learning. The learning unit expands learning data by integrating information from different data sources during learning. The integration is performed, for example, by collecting and integrating data, but is not limited to such examples. For example, the learning unit integrates emotional data from different data sources for learning. The learning unit can also expand learning data based on information from different data sources. The learning unit can also use data from different data sources to improve the accuracy of the learning algorithm. Thus, by integrating information from different data sources, learning data can be expanded and the accuracy of learning can be improved. Some or all of the above-described processing in the learning unit may be performed using AI, or may be performed without using AI. For example, the learning unit can input information from different data sources into an AI model, and the AI can perform data integration.

The emotion recognition support system comprises a learning unit that adjusts the learning algorithm by considering the subject's social background during learning. The learning unit adjusts the learning algorithm by considering the subject's social background (such as culture, customs, etc.) during learning. The adjustment is performed, for example, by setting algorithm parameters or weighting, but is not limited to such examples. For example, the learning unit adjusts the learning algorithm by considering the subject's cultural background. The learning unit can also adjust the learning algorithm by considering the subject's customs and lifestyle. The learning unit can also optimize the learning algorithm based on the subject's social background. Thus, by considering social background, the learning algorithm can be optimized and the accuracy of learning can be improved. Some or all of the above-described processing in the learning unit may be performed using AI, or may be performed without using AI. For example, the learning unit can input social background data into an AI model, and the AI can perform adjustment of the algorithm.

Below is a brief explanation of the processing flow of Example 1 of the Embodiment.
Step 1: The collection unit collects the facial expressions and voice tone of the subject. For example, the collection unit uses a microphone-camera type device to collect facial expressions such as smiles, anger, sadness, and voice characteristics such as high pitch, low pitch, and intonation.
Step 2: The analysis unit analyzes the information collected by the collection unit and identifies the emotions of the subject. For example, the analysis unit uses an emotion recognition algorithm to analyze facial expression data and voice tone, and determines whether the subject is smiling, angry, etc.
Step 3: The provision unit provides the analysis results obtained by the analysis unit in real time. For example, the provision unit can provide the results to caregivers or child welfare workers through an earphone-type device, display them on a display, or notify them by voice.
Step 4: The learning unit performs self-learning to respond to the individual care needs of users based on the information provided by the provision unit. For example, the learning unit uses a machine learning algorithm to learn what kind of emotions a specific subject shows in specific situations and what kind of responses are preferred, and presents the optimal approach.

### Example 2 of the Embodiment

The emotion recognition support system according to the embodiment of the present invention is a system that collects the facial expressions and voice tone of a subject, analyzes these pieces of information using AI to identify emotions, and provides the results in real time. The emotion recognition support system collects the facial expressions and voice tone of the subject, and AI analyzes these pieces of information to identify emotions. The analysis results are provided in real time through an earphone-type device, and appropriate countermeasures are presented. Furthermore, the AI performs self-learning to respond to the individual care needs of each user and presents the optimal approach. For example, the emotion recognition support system collects the facial expressions and voice tone of the subject using a microphone-camera type device. For example, if the subject is speaking with a smile, the system collects that facial expression and voice tone. This information is input to the AI. Next, the emotion recognition support system analyzes the collected information using AI to identify the emotions of the subject. For example, the AI analyzes the smile and bright voice tone and determines that the subject is happy. This analysis result is provided in real time through an earphone-type device. Furthermore, the emotion recognition support system performs self-learning with AI so as to respond to the individual care needs of each user. For example, it learns what kind of emotions a specific subject shows in specific situations and presents the optimal approach based on that information. As a result, the emotion recognition support system can reduce the burden on caregivers and child welfare workers and improve the quality of welfare services. By enabling real-time understanding of the subject's emotions, appropriate responses can be made quickly, thereby reducing the stress of caregivers and child welfare workers. In addition, by responding based on the subject's emotions, the satisfaction of the subject is improved. Furthermore, by having AI perform emotion analysis and present countermeasures, the work of caregivers and child welfare workers is streamlined, and high-quality welfare services can be provided even with a small workforce.

The emotion recognition support system according to the embodiment comprises a collection unit, an analysis unit, a provision unit, and a learning unit. The collection unit collects the facial expressions and voice tone of the subject. The facial expressions of the subject may include, for example, smiles, anger, sadness, etc., but are not limited to such examples. The collection unit collects the facial expressions and voice tone of the subject using, for example, a microphone-camera type device. The collection unit can also collect the voice tone of the subject. For example, the collection unit collects the high pitch, low pitch, and intonation of the subject's voice. The analysis unit analyzes the information collected by the collection unit and identifies the emotions of the subject. The identification of emotions is performed, for example, using an emotion recognition algorithm, but is not limited to such examples. For example, the analysis unit analyzes the collected facial expression data and determines whether the subject is smiling. The analysis unit can also analyze the collected voice tone and determine whether the subject is angry. The analysis unit can also analyze the collected facial expression data and voice tone in combination to comprehensively identify the emotions of the subject. The provision unit provides the analysis results obtained by the analysis unit in real time. The provision may be performed, for example, through an earphone-type device, but is not limited to such examples. For example, the provision unit provides the analysis results to caregivers or child welfare workers through an earphone-type device. The provision unit can also display the analysis results on a display. The provision unit can also notify the analysis results by voice. The learning unit performs self-learning to respond to the individual care needs of users based on the information provided by the provision unit. The self-learning is performed, for example, using a machine learning algorithm, but is not limited to such examples. For example, the learning unit learns what kind of emotions a specific subject shows in specific situations. The learning unit can also learn what kind of responses a specific subject prefers in specific situations. The learning unit can also present the optimal approach based on the learned information. As a result, the emotion recognition support system according to the embodiment can reduce the burden on caregivers and child welfare workers and improve the quality of welfare services.

The emotion recognition support system comprises a collection unit that collects the facial expressions or voice tone of the subject using a microphone-camera type device. The collection unit collects the facial expressions or voice tone of the subject using a microphone-camera type device. The microphone-camera type device may include, for example, a high-resolution camera or a high-sensitivity microphone, but is not limited to such examples. For example, the collection unit collects the facial expressions of the subject in detail using a high-resolution camera. The collection unit can also accurately collect the voice tone of the subject using a high-sensitivity microphone. The collection unit can also collect the natural facial expressions and voice tone of the subject by devising the installation method of the microphone-camera type device. For example, the collection unit installs the camera at the eye level of the subject to collect natural facial expressions. The collection unit can also install the microphone close to the subject's mouth to accurately collect the voice tone. Thus, by using a microphone-camera type device, the facial expressions and voice tone of the subject can be accurately collected.

The analysis unit can analyze the collected information and identify the emotions of the subject. The analysis unit analyzes the collected information and identifies the emotions of the subject. The analysis is performed, for example, using a data analysis algorithm, but is not limited to such examples. For example, the analysis unit analyzes the collected facial expression data and determines whether the subject is smiling. The analysis unit can also analyze the collected voice tone and determine whether the subject is angry. The analysis unit can also analyze the collected facial expression data and voice tone in combination to comprehensively identify the emotions of the subject. For example, the analysis unit analyzes the facial expressions of the subject using a facial recognition algorithm. The analysis unit can also analyze the voice tone of the subject using a voice analysis algorithm. The analysis unit can also use an emotion recognition algorithm to analyze the combination of facial expression data and voice tone to identify the emotions of the subject. Thus, by analyzing the collected information, the emotions of the subject can be accurately identified.

The provision unit can provide the analysis results in real time through an earphone-type device. The provision unit provides the analysis results in real time through an earphone-type device. The earphone-type device may include, for example, high-quality earphones or wireless earphones, but is not limited to such examples. For example, the provision unit provides the analysis results clearly using high-quality earphones. The provision unit can also provide the analysis results using wireless earphones. The provision unit can also provide the analysis results in real time by having caregivers or child welfare workers wear the earphone-type device. For example, the provision unit notifies the analysis results by voice. The provision unit can also notify the analysis results by text message. The provision unit can also display the analysis results on a display. Thus, by providing the analysis results in real time through an earphone-type device, caregivers and child welfare workers can respond quickly.

The learning unit can learn the emotional patterns of a specific subject and present the optimal approach. The learning unit learns the emotional patterns of a specific subject and presents the optimal approach. The learning is performed, for example, using a machine learning algorithm, but is not limited to such examples. For example, the learning unit learns what kind of emotions a specific subject shows in specific situations. The learning unit can also learn what kind of responses a specific subject prefers in specific situations. The learning unit can also present the optimal approach based on the learned information. For example, if the specific subject is feeling stressed, the learning unit presents an approach with a relaxing effect. If the specific subject is relaxed, the learning unit can present an approach that provides detailed information. If the specific subject is excited, the learning unit can present a visually stimulating approach. Thus, by learning the emotional patterns of a specific subject, the optimal approach tailored to individual care needs can be provided.

The provision unit can aim to reduce the burden on caregivers and child welfare workers and improve the quality of welfare services. The provision unit aims to reduce the burden on caregivers and child welfare workers and improve the quality of welfare services. The burden reduction is performed, for example, by shortening work time or reducing stress, but is not limited to such examples. For example, the provision unit shortens the work time of caregivers and child welfare workers by providing emotion analysis results in real time. The provision unit can also reduce the stress of caregivers and child welfare workers by presenting appropriate countermeasures. The provision unit also aims to improve the quality of welfare services. The quality improvement is performed, for example, through service evaluation criteria or improvement methods, but is not limited to such examples. For example, the provision unit sets service evaluation criteria based on emotion analysis results. The provision unit can also propose service improvement methods based on emotion analysis results. Thus, the burden on caregivers and child welfare workers can be reduced and the quality of welfare services can be improved.

The emotion recognition support system comprises a collection unit that estimates the user's emotions and determines the priority of collecting facial expressions or voice tone to be collected based on the estimated emotions of the user. The collection unit estimates the user's emotions and determines the priority of collecting facial expressions or voice tone to be collected based on the estimated emotions of the user. The determination of priority is performed, for example, based on importance or urgency, but is not limited to such examples. For example, if the user is feeling stressed, the collection unit prioritizes collecting subtle changes in facial expressions. If the user is relaxed, the collection unit can prioritize collecting changes in voice tone. If the user is excited, the collection unit can collect both facial expressions and voice tone in a balanced manner. Thus, by determining the priority of data to be collected based on the user's emotions, more accurate emotion analysis becomes possible. Some or all of the above-described processing in the collection unit may be performed using AI, or may be performed without using AI. For example, the collection unit can use an AI model to analyze facial expressions and voice tone to estimate the user's emotions and determine the priority of data to be collected based on the results.

The emotion recognition support system comprises a collection unit that optimizes the collection method by referring to the subject's past emotional data at the time of collection. The collection unit optimizes the collection method by referring to the subject's past emotional data at the time of collection. The optimization is performed, for example, by adjusting algorithms or setting parameters, but is not limited to such examples. For example, the collection unit refers to situations in which the subject felt stressed in the past and focuses on collecting facial expressions and voice tone in similar situations. The collection unit can also refer to situations in which the subject was relaxed in the past and adjust the collection method based on the data from that time. The collection unit can also refer to situations in which the subject was excited in the past and optimize the collection method based on the data from that time. Thus, by referring to past emotional data, the collection method can be optimized and more accurate data collection becomes possible. Some or all of the above-described processing in the collection unit may be performed using AI, or may be performed without using AI. For example, the collection unit can input past emotional data into an AI model, and the AI can propose the optimal collection method.

The emotion recognition support system comprises a collection unit that adjusts the collection means based on the subject's current environment at the time of collection. The collection unit adjusts the collection means based on the subject's current environment (indoors/outdoors, noise level, etc.) at the time of collection. The adjustment is performed, for example, by selecting the collection means or changing settings, but is not limited to such examples. For example, if the environment is quiet indoors, the collection unit collects subtle changes in voice tone. If the environment is noisy outdoors, the collection unit can prioritize collecting changes in facial expressions. If the noise level is moderate, the collection unit can collect both facial expressions and voice tone in a balanced manner. Thus, by adjusting the collection means based on the current environment, more appropriate data collection becomes possible. Some or all of the above-described processing in the collection unit may be performed using AI, or may be performed without using AI. For example, the collection unit can input current environment data into an AI model, and the AI can propose the optimal collection means.

The emotion recognition support system comprises a collection unit that corrects the collected data by considering the subject's physical condition at the time of collection. The collection unit corrects the collected data by considering the subject's physical condition (fatigue, health status, etc.) at the time of collection. The correction is performed, for example, by adjusting or filtering the data, but is not limited to such examples. For example, if the subject is tired, the collection unit focuses on collecting changes in facial expressions and corrects changes in voice tone. If the subject is in good health, the collection unit can collect both facial expressions and voice tone in a balanced manner. If the subject is in poor physical condition, the collection unit can focus on collecting changes in voice tone and correct changes in facial expressions. Thus, by correcting the collected data considering the physical condition, more accurate data collection becomes possible. Some or all of the above-described processing in the collection unit may be performed using AI, or may be performed without using AI. For example, the collection unit can input physical condition data into an AI model, and the AI can perform the correction of the collected data.

The emotion recognition support system comprises a collection unit that estimates the user's emotions and filters the data to be collected based on the estimated emotions of the user. The collection unit estimates the user's emotions and filters the data to be collected based on the estimated emotions of the user. The filtering is performed, for example, by selecting data or removing noise, but is not limited to such examples. For example, if the user is feeling stressed, the collection unit removes noise and collects subtle changes in facial expressions. If the user is relaxed, the collection unit can remove background noise and collect changes in voice tone. If the user is excited, the collection unit can filter and collect both facial expressions and voice tone. Thus, by filtering data based on the user's emotions, more accurate emotion analysis becomes possible. Some or all of the above-described processing in the collection unit may be performed using AI, or may be performed without using AI. For example, the collection unit can input the user's emotional data into an AI model, and the AI can perform filtering.

The emotion recognition support system comprises a collection unit that enhances the relevance of collected data by considering the subject's geographic location information at the time of collection. The collection unit enhances the relevance of collected data by considering the subject's geographic location information at the time of collection. The enhancement of relevance is performed, for example, by selecting or filtering data, but is not limited to such examples. For example, if the subject is in a park, the collection unit removes natural sounds and collects facial expressions and voice tone. If the subject is in an office, the collection unit can remove background conversation sounds and collect facial expressions and voice tone. If the subject is at home, the collection unit can remove household noise and collect facial expressions and voice tone. Thus, by considering geographic location information, the relevance of collected data can be enhanced and more accurate data collection becomes possible. Some or all of the above-described processing in the collection unit may be performed using AI, or may be performed without using AI. For example, the collection unit can input geographic location information into an AI model, and the AI can perform filtering to enhance the relevance of collected data.

The emotion recognition support system comprises a collection unit that analyzes the subject's social media activity at the time of collection and supplements relevant emotional data. The collection unit analyzes the subject's social media activity at the time of collection and supplements relevant emotional data. The supplementation is performed, for example, by adding or correcting data, but is not limited to such examples. For example, the collection unit analyzes the content of the subject's social media posts and supplements changes in emotions. The collection unit can also refer to the activities of the subject's friends on social media to supplement emotional data. The collection unit can also supplement emotional data based on the subject's check-in information on social media. Thus, by analyzing social media activity, relevant emotional data can be supplemented and more accurate emotion analysis becomes possible. Some or all of the above-described processing in the collection unit may be performed using AI, or may be performed without using AI. For example, the collection unit can input social media activity data into an AI model, and the AI can perform supplementation of emotional data.

The emotion recognition support system comprises a collection unit that customizes the collection method by reflecting the subject's past feedback at the time of collection. The collection unit customizes the collection method by reflecting the subject's past feedback at the time of collection. The customization is performed, for example, by selecting the collection means or changing settings, but is not limited to such examples. For example, the collection unit adjusts the collection method based on feedback previously provided by the subject. The collection unit can also preferentially use collection methods that the subject has preferred in the past. The collection unit can also analyze the subject's past feedback and propose the optimal collection method. Thus, by reflecting past feedback, the collection method can be customized and more accurate data collection becomes possible. Some or all of the above-described processing in the collection unit may be performed using AI, or may be performed without using AI. For example, the collection unit can input past feedback data into an AI model, and the AI can perform customization of the collection method.

The emotion recognition support system comprises an analysis unit that estimates the user's emotions and adjusts the analysis algorithm based on the estimated emotions of the user. The analysis unit estimates the user's emotions and adjusts the analysis algorithm based on the estimated emotions of the user. The adjustment is performed, for example, by setting algorithm parameters or weighting, but is not limited to such examples. For example, if the user is feeling stressed, the analysis unit uses an algorithm that emphasizes subtle changes in emotions. If the user is relaxed, the analysis unit can use an algorithm that emphasizes the overall tone of emotions. If the user is excited, the analysis unit can use an algorithm that emphasizes rapid changes in emotions. Thus, by adjusting the analysis algorithm based on the user's emotions, more accurate emotion analysis becomes possible. Some or all of the above-described processing in the analysis unit may be performed using AI, or may be performed without using AI. For example, the analysis unit can input the user's emotional data into an AI model, and the AI can perform adjustment of the analysis algorithm.

The emotion recognition support system comprises an analysis unit that improves accuracy by comparing the subject's emotional patterns with past data during analysis. The analysis unit improves accuracy by comparing the subject's emotional patterns with past data during analysis. The improvement of accuracy is performed, for example, by improving data accuracy or algorithms, but is not limited to such examples. For example, the analysis unit analyzes by comparing the current emotional patterns with the subject's past emotional data. The analysis unit can also refer to the subject's past emotional patterns to more accurately identify current emotions. The analysis unit can also use the subject's past emotional data to improve the accuracy of the analysis algorithm. Thus, by comparing with past data, the accuracy of emotion analysis can be improved. Some or all of the above-described processing in the analysis unit may be performed using AI, or may be performed without using AI. For example, the analysis unit can input past emotional data into an AI model, and the AI can perform analysis for accuracy improvement.

The emotion recognition support system comprises an analysis unit that tracks fluctuations in the subject's emotions in real time during analysis and updates the analysis results. The analysis unit tracks fluctuations in the subject's emotions in real time during analysis and updates the analysis results. The tracking is performed, for example, by monitoring emotional fluctuations in real time and updating the analysis results each time a fluctuation occurs, but is not limited to such examples. For example, the analysis unit updates the analysis results in real time each time the subject's emotions fluctuate. The analysis unit can also track fluctuations in the subject's emotions in real time and provide the latest analysis results. The analysis unit can also immediately update the analysis results if the subject's emotions fluctuate rapidly. Thus, by tracking emotional fluctuations in real time, the latest analysis results can be provided. Some or all of the above-described processing in the analysis unit may be performed using AI, or may be performed without using AI. For example, the analysis unit can input real-time emotional data into an AI model, and the AI can perform updating of the analysis results.

The emotion recognition support system comprises an analysis unit that considers external factors affecting the subject's emotions during analysis. The analysis unit considers external factors (such as weather, time of day, etc.) affecting the subject's emotions during analysis. The consideration is performed, for example, by collecting data on external factors and reflecting them in emotion analysis, but is not limited to such examples. For example, the analysis unit analyzes by considering the impact of bad weather on the subject's emotions. The analysis unit can also analyze by considering emotional fluctuations due to time of day. The analysis unit can also analyze by considering external factors (such as noise level, influence of surrounding people, etc.). Thus, by considering external factors, more accurate emotion analysis becomes possible. Some or all of the above-described processing in the analysis unit may be performed using AI, or may be performed without using AI. For example, the analysis unit can input external factor data into an AI model and reflect it in emotion analysis.

The emotion recognition support system comprises an analysis unit that estimates the user's emotions and adjusts the display method of analysis results based on the estimated emotions of the user. The analysis unit estimates the user's emotions and adjusts the display method of analysis results based on the estimated emotions of the user. The adjustment is performed, for example, by selecting the display format or changing settings, but is not limited to such examples. For example, if the user is feeling stressed, the analysis unit provides a simple and highly visible display method. If the user is relaxed, the analysis unit can provide a display method that includes detailed information. If the user is excited, the analysis unit can provide a visually stimulating display method. Thus, by adjusting the display method based on the user's emotions, more appropriate information provision becomes possible. Some or all of the above-described processing in the analysis unit may be performed using AI, or may be performed without using AI. For example, the analysis unit can input the user's emotional data into an AI model, and the AI can perform adjustment of the display method.

The emotion recognition support system comprises an analysis unit that performs emotion analysis by considering the subject's geographic background during analysis. The analysis unit performs emotion analysis by considering the subject's geographic background during analysis. The consideration is performed, for example, by collecting data on geographic background and reflecting it in emotion analysis, but is not limited to such examples. For example, if the subject is in an urban area, the analysis unit analyzes by considering urban-specific stress factors. If the subject is in a natural environment, the analysis unit can analyze by considering relaxation effects. If the subject is in a specific region, the analysis unit can analyze by considering the culture and customs specific to that region. Thus, by considering geographic background, more accurate emotion analysis becomes possible. Some or all of the above-described processing in the analysis unit may be performed using AI, or may be performed without using AI. For example, the analysis unit can input geographic background data into an AI model and reflect it in emotion analysis.

The emotion recognition support system comprises an analysis unit that improves analysis accuracy by referring to relevant literature and research data on the subject during analysis. The analysis unit improves analysis accuracy by referring to relevant literature and research data on the subject during analysis. The reference is performed, for example, by collecting and analyzing relevant literature and research data, but is not limited to such examples. For example, the analysis unit analyzes by referring to the latest research data on the subject's emotions. The analysis unit can also adjust the analysis algorithm based on relevant literature on the subject's emotions. The analysis unit can also improve analysis accuracy by referring to past research data on the subject's emotions. Thus, by referring to relevant literature and research data, analysis accuracy can be improved. Some or all of the above-described processing in the analysis unit may be performed using AI, or may be performed without using AI. For example, the analysis unit can input relevant literature and research data into an AI model, and the AI can perform analysis accuracy improvement.

The emotion recognition support system comprises an analysis unit that analyzes emotions by considering the subject's social background during analysis. The analysis unit analyzes emotions by considering the subject's social background (such as culture, customs, etc.) during analysis. The consideration is performed, for example, by collecting data on social background and reflecting it in emotion analysis, but is not limited to such examples. For example, the analysis unit analyzes emotions by considering the subject's cultural background. The analysis unit can also analyze emotions by considering the subject's customs and lifestyle. The analysis unit can also analyze emotional fluctuations based on the subject's social background. Thus, by considering social background, more accurate emotion analysis becomes possible. Some or all of the above-described processing in the analysis unit may be performed using AI, or may be performed without using AI. For example, the analysis unit can input social background data into an AI model and reflect it in emotion analysis.

The emotion recognition support system comprises a provision unit that estimates the user's emotions and determines the priority of information to be provided based on the estimated emotions of the user. The provision unit estimates the user's emotions and determines the priority of information to be provided based on the estimated emotions of the user. The determination of priority is performed, for example, based on the importance or urgency of the information, but is not limited to such examples. For example, if the user is feeling stressed, the provision unit prioritizes providing information with a relaxing effect. If the user is relaxed, the provision unit can prioritize providing detailed information. If the user is excited, the provision unit can prioritize providing visually stimulating information. Thus, by determining the priority of information based on the user's emotions, more appropriate information provision becomes possible. Some or all of the above-described processing in the provision unit may be performed using AI, or may be performed without using AI. For example, the provision unit can input the user's emotional data into an AI model, and the AI can perform determination of information priority.

The emotion recognition support system comprises a provision unit that optimizes the information provision method by referring to the subject's past responses at the time of provision. The provision unit optimizes the information provision method by referring to the subject's past responses at the time of provision. The optimization is performed, for example, by selecting the information provision means or changing settings, but is not limited to such examples. For example, the provision unit preferentially uses information provision methods that the subject has preferred in the past. The provision unit can also propose the optimal information provision method based on the subject's past responses. The provision unit can also analyze the subject's past responses and optimize the information provision method. Thus, by referring to past responses, the information provision method can be optimized and more appropriate information provision becomes possible. Some or all of the above-described processing in the provision unit may be performed using AI, or may be performed without using AI. For example, the provision unit can input past response data into an AI model, and the AI can perform optimization of the information provision method.

The emotion recognition support system comprises a provision unit that adjusts information provision based on the subject's current situation at the time of provision. The provision unit adjusts information provision based on the subject's current situation (such as being active or on break) at the time of provision. The adjustment is performed, for example, by changing the timing or format of information provision, but is not limited to such examples. For example, if the subject is active, the provision unit provides concise and to-the-point information. If the subject is on break, the provision unit can provide detailed information. The provision unit can also propose the optimal information provision method based on the subject's current situation. Thus, by adjusting information provision based on the current situation, more appropriate information provision becomes possible. Some or all of the above-described processing in the provision unit may be performed using AI, or may be performed without using AI. For example, the provision unit can input current situation data into an AI model, and the AI can perform adjustment of information provision.

The emotion recognition support system comprises a provision unit that corrects information provision by considering the subject's physical condition at the time of provision. The provision unit corrects information provision by considering the subject's physical condition (such as fatigue, health status, etc.) at the time of provision. The correction is performed, for example, by changing the content or format of information, but is not limited to such examples. For example, if the subject is tired, the provision unit provides concise and to-the-point information. If the subject is in good health, the provision unit can provide detailed information. If the subject is in poor physical condition, the provision unit can provide information with a relaxing effect. Thus, by correcting information provision considering the physical condition, more appropriate information provision becomes possible. Some or all of the above-described processing in the provision unit may be performed using AI, or may be performed without using AI. For example, the provision unit can input physical condition data into an AI model, and the AI can perform correction of information provision.

The emotion recognition support system comprises a provision unit that estimates the user's emotions and adjusts the format of information to be provided based on the estimated emotions of the user. The provision unit estimates the user's emotions and adjusts the format of information to be provided based on the estimated emotions of the user. The adjustment is performed, for example, by changing the display format or notification method of information, but is not limited to such examples. For example, if the user is feeling stressed, the provision unit provides information in a visually simple format. If the user is relaxed, the provision unit can provide information in a format that includes detailed graphics or charts. If the user is excited, the provision unit can provide information in a visually stimulating format. Thus, by adjusting the format of information based on the user's emotions, more appropriate information provision becomes possible. Some or all of the above-described processing in the provision unit may be performed using AI, or may be performed without using AI. For example, the provision unit can input the user's emotional data into an AI model, and the AI can perform adjustment of the information format.

The emotion recognition support system comprises a provision unit that provides highly relevant information by considering the subject's geographic location information at the time of provision. The provision unit provides highly relevant information by considering the subject's geographic location information at the time of provision. The enhancement of relevance is performed, for example, by selecting or filtering information, but is not limited to such examples. For example, if the subject is in a specific region, the provision unit provides information related to that region. If the subject is traveling, the provision unit can provide information related to the travel destination. If the subject is at home, the provision unit can provide information about the area around the home. Thus, by considering geographic location information, highly relevant information provision becomes possible. Some or all of the above-described processing in the provision unit may be performed using AI, or may be performed without using AI. For example, the provision unit can input geographic location information into an AI model, and the AI can select highly relevant information.

The emotion recognition support system comprises a provision unit that analyzes the subject's social media activity at the time of provision and provides relevant information. The provision unit analyzes the subject's social media activity at the time of provision and provides relevant information. The analysis is performed, for example, by analyzing the content and reactions of social media posts, but is not limited to such examples. For example, the provision unit provides information about places where the subject has checked in on social media. The provision unit can also analyze the content of the subject's social media posts and provide relevant information. The provision unit can also refer to the activities of the subject's friends on social media to provide relevant information. Thus, by analyzing social media activity, relevant information provision becomes possible. Some or all of the above-described processing in the provision unit may be performed using AI, or may be performed without using AI. For example, the provision unit can input social media activity data into an AI model, and the AI can select relevant information.

The emotion recognition support system comprises a provision unit that customizes the information provision method by reflecting the subject's past feedback at the time of provision. The provision unit customizes the information provision method by reflecting the subject's past feedback at the time of provision. The customization is performed, for example, by selecting the information provision means or changing settings, but is not limited to such examples. For example, the provision unit preferentially uses information provision methods that the subject has preferred in the past. The provision unit can also propose the optimal information provision method based on the subject's past feedback. The provision unit can also analyze the subject's past feedback and customize the information provision method. Thus, by reflecting past feedback, the information provision method can be customized and more appropriate information provision becomes possible. Some or all of the above-described processing in the provision unit may be performed using AI, or may be performed without using AI. For example, the provision unit can input past feedback data into an AI model, and the AI can perform customization of the information provision method.

The emotion recognition support system comprises a learning unit that estimates the user's emotions and selects learning data based on the estimated emotions of the user. The learning unit estimates the user's emotions and selects learning data based on the estimated emotions of the user. The selection is performed, for example, by determining the type or priority of learning data, but is not limited to such examples. For example, if the user is feeling stressed, the learning unit prioritizes learning data related to stress reduction. If the user is relaxed, the learning unit can prioritize learning data with a relaxing effect. If the user is excited, the learning unit can prioritize learning data that calms excitement. Thus, by selecting learning data based on the user's emotions, more effective learning becomes possible. Some or all of the above-described processing in the learning unit may be performed using AI, or may be performed without using AI. For example, the learning unit can input the user's emotional data into an AI model, and the AI can perform selection of learning data.

The emotion recognition support system comprises a learning unit that optimizes the learning algorithm by referring to past learning data during learning. The learning unit optimizes the learning algorithm by referring to past learning data during learning. The optimization is performed, for example, by adjusting algorithms or setting parameters, but is not limited to such examples. For example, the learning unit adjusts the current learning algorithm based on past learning data. The learning unit can also refer to past learning data to find the optimal learning pattern. The learning unit can also use past learning data to improve the accuracy of the learning algorithm. Thus, by referring to past learning data, the learning algorithm can be optimized and the accuracy of learning can be improved. Some or all of the above-described processing in the learning unit may be performed using AI, or may be performed without using AI. For example, the learning unit can input past learning data into an AI model, and the AI can perform optimization of the learning algorithm.

The emotion recognition support system comprises a learning unit that analyzes fluctuations in the subject's emotional patterns during learning and adjusts the update frequency of learning data. The learning unit analyzes fluctuations in the subject's emotional patterns during learning and adjusts the update frequency of learning data. The adjustment is performed, for example, by setting the update frequency or weighting data, but is not limited to such examples. For example, if the subject's emotional patterns fluctuate frequently, the learning unit increases the update frequency of learning data. If the subject's emotional patterns are stable, the learning unit can decrease the update frequency of learning data. The learning unit can also analyze fluctuations in the subject's emotional patterns and set the optimal update frequency. Thus, by analyzing fluctuations in emotional patterns, the update frequency of learning data can be optimized and the accuracy of learning can be improved. Some or all of the above-described processing in the learning unit may be performed using AI, or may be performed without using AI. For example, the learning unit can input fluctuation data of emotional patterns into an AI model, and the AI can perform adjustment of the update frequency.

The emotion recognition support system comprises a learning unit that updates learning data by reflecting the subject's feedback during learning. The learning unit updates learning data by reflecting the subject's feedback during learning. The update is performed, for example, by collecting and analyzing feedback, but is not limited to such examples. For example, the learning unit updates learning data based on feedback provided by the subject. The learning unit can also analyze the subject's feedback and adjust the learning algorithm. The learning unit can also improve the accuracy of learning data by reflecting the subject's feedback. Thus, by reflecting feedback, learning data can be updated and the accuracy of learning can be improved. Some or all of the above-described processing in the learning unit may be performed using AI, or may be performed without using AI. For example, the learning unit can input feedback data into an AI model, and the AI can perform updating of learning data.

The emotion recognition support system comprises a learning unit that estimates the user's emotions and adjusts the frequency of learning based on the estimated emotions of the user. The learning unit estimates the user's emotions and adjusts the frequency of learning based on the estimated emotions of the user. The adjustment is performed, for example, by setting the timing or frequency of learning, but is not limited to such examples. For example, if the user is feeling stressed, the learning unit increases the frequency of learning. If the user is relaxed, the learning unit can decrease the frequency of learning. If the user is excited, the learning unit can adjust the frequency of learning. Thus, by adjusting the frequency of learning based on the user's emotions, more effective learning becomes possible. Some or all of the above-described processing in the learning unit may be performed using AI, or may be performed without using AI. For example, the learning unit can input the user's emotional data into an AI model, and the AI can perform adjustment of the learning frequency.

The emotion recognition support system comprises a learning unit that weights learning data based on the submission timing of the subject's emotional data during learning. The learning unit weights learning data based on the submission timing of the subject's emotional data during learning. The weighting is performed, for example, based on the importance or freshness of the data, but is not limited to such examples. For example, the learning unit weights learning data based on the timing when the subject submitted emotional data. The learning unit can also adjust the learning algorithm by considering the submission timing of the subject's emotional data. The learning unit can also set the priority of learning data based on the submission timing of the subject's emotional data. Thus, by weighting learning data based on the submission timing of emotional data, the accuracy of learning can be improved. Some or all of the above-described processing in the learning unit may be performed using AI, or may be performed without using AI. For example, the learning unit can input the submission timing of emotional data into an AI model, and the AI can perform

The emotion recognition support system comprises a learning unit that expands learning data by integrating information from different data sources during learning. The learning unit expands learning data by integrating information from different data sources during learning. The integration is performed, for example, by collecting and integrating data, but is not limited to such examples. For example, the learning unit integrates emotional data from different data sources for learning. The learning unit can also expand learning data based on information from different data sources. The learning unit can also use data from different data sources to improve the accuracy of the learning algorithm. Thus, by integrating information from different data sources, learning data can be expanded and the accuracy of learning can be improved. Some or all of the above-described processing in the learning unit may be performed using AI, or may be performed without using AI. For example, the learning unit can input information from different data sources into an AI model, and the AI can perform data integration.

The emotion recognition support system comprises a learning unit that adjusts the learning algorithm by considering the subject's social background during learning. The learning unit adjusts the learning algorithm by considering the subject's social background (such as culture, customs, etc.) during learning. The adjustment is performed, for example, by setting algorithm parameters or weighting, but is not limited to such examples. For example, the learning unit adjusts the learning algorithm by considering the subject's cultural background. The learning unit can also adjust the learning algorithm by considering the subject's customs and lifestyle. The learning unit can also optimize the learning algorithm based on the subject's social background. Thus, by considering social background, the learning algorithm can be optimized and the accuracy of learning can be improved. Some or all of the above-described processing in the learning unit may be performed using AI, or may be performed without using AI. For example, the learning unit can input social background data into an AI model, and the AI can perform adjustment of the algorithm.

### ===Hardware Guarantee 1-1===

Each of the multiple elements including the above-described collection unit, analysis unit, provision unit, and learning unit is implemented, for example, by at least one of the smart device 14 and the data processing device 12. For example, the collection unit collects the facial expressions and voice tone of the subject using the camera 42 or microphone 38B of the smart device 14. The analysis unit is implemented, for example, by the specific processing unit 290 of the data processing device 12, and analyzes the collected information to identify emotions. The provision unit is implemented, for example, by the control unit 46A of the smart device 14, and provides the analysis results in real time through an earphone-type device. The learning unit is implemented, for example, by the specific processing unit 290 of the data processing device 12, and performs self-learning to respond to the individual care needs of users.

### ===Hardware Guarantee 1-2===

Each of the multiple elements including the above-described collection unit, analysis unit, provision unit, and learning unit is implemented, for example, by at least one of the smart glasses 214 and the data processing device 12. For example, the collection unit collects the facial expressions and voice tone of the subject using the camera 42 or microphone 238 of the smart glasses 214. The analysis unit is implemented, for example, by the specific processing unit 290 of the data processing device 12, and analyzes the collected information to identify emotions. The provision unit is implemented, for example, by the control unit 46A of the smart glasses 214, and provides the analysis results in real time through an earphone-type device. The learning unit is implemented, for example, by the specific processing unit 290 of the data processing device 12, and performs self-learning to respond to the individual care needs of users.

### ===Hardware Guarantee 1-3===

Each of the multiple elements including the above-described collection unit, analysis unit, provision unit, and learning unit is implemented, for example, by at least one of the headset-type terminal 314 and the data processing device 12. For example, the collection unit collects the facial expressions and voice tone of the subject using the camera 42 or microphone 238 of the headset-type terminal 314. The analysis unit is implemented, for example, by the specific processing unit 290 of the data processing device 12, and analyzes the collected information to identify emotions. The provision unit is implemented, for example, by the control unit 46A of the headset-type terminal 314, and provides the analysis results in real time through an earphone-type device. The learning unit is implemented, for example, by the specific processing unit 290 of the data processing device 12, and performs self-learning to respond to the individual care needs of users.

### ===Hardware Guarantee 1-4===

Each of the multiple elements including the above-described collection unit, analysis unit, provision unit, and learning unit is implemented, for example, by at least one of the robot 414 and the data processing device 12. For example, the collection unit collects the facial expressions and voice tone of the subject using the camera 42 or microphone 238 of the robot 414. The analysis unit is implemented, for example, by the specific processing unit 290 of the data processing device 12, and analyzes the collected information to identify emotions. The provision unit is implemented, for example, by the control unit 46A of the robot 414, and provides the analysis results in real time through an earphone-type device. The learning unit is implemented, for example, by the specific processing unit 290 of the data processing device 12, and performs self-learning to respond to the individual care needs of users.

The system according to the embodiment is not limited to the examples described above, and various modifications are possible, for example, as follows.

The emotion recognition support system comprises a collection unit that estimates the user's emotions and determines the priority of collecting facial expressions or voice tone to be collected based on the estimated emotions of the user. The collection unit estimates the user's emotions and determines the priority of collecting facial expressions or voice tone to be collected based on the estimated emotions of the user. The determination of priority is performed, for example, based on importance or urgency, but is not limited to such examples. For example, if the user is feeling stressed, the collection unit prioritizes collecting subtle changes in facial expressions. Additionally, if the user is relaxed, the collection unit may prioritize collecting changes in voice tone. Furthermore, if the user is excited, the collection unit may collect both facial expressions and voice tone in a balanced manner. Thus, by determining the priority of data to be collected based on the user's emotions, more accurate emotion analysis becomes possible. Some or all of the above-described processing in the collection unit may be performed using AI, or may be performed without using AI. For example, the collection unit may analyze facial expressions and voice tone using an AI model to estimate the user's emotions and determine the priority of data to be collected based on the results.

The emotion recognition support system comprises a collection unit that optimizes the collection method by referring to the subject's past emotional data at the time of collection. The collection unit optimizes the collection method by referring to the subject's past emotional data at the time of collection. The optimization is performed, for example, by adjusting algorithms or setting parameters, but is not limited to such examples. For example, the collection unit refers to situations in which the subject felt stressed in the past and focuses on collecting facial expressions and voice tone in similar situations. The collection unit can also refer to situations in which the subject was relaxed in the past and adjust the collection method based on the data from that time. The collection unit can also refer to situations in which the subject was excited in the past and optimize the collection method based on the data from that time. Thus, by referring to past emotional data, the collection method can be optimized and more accurate data collection becomes possible. Some or all of the above-described processing in the collection unit may be performed using AI, or may be performed without using AI. For example, the collection unit can input past emotional data into an AI model, and the AI can propose the optimal collection method.

The emotion recognition support system comprises a collection unit that adjusts the collection means based on the subject's current environment at the time of collection. The collection unit adjusts the collection means based on the subject's current environment (indoors/outdoors, noise level, etc.) at the time of collection. The adjustment is performed, for example, by selecting the collection means or changing settings, but is not limited to such examples. For example, if the environment is quiet indoors, the collection unit collects subtle changes in voice tone. If the environment is noisy outdoors, the collection unit can prioritize collecting changes in facial expressions. If the noise level is moderate, the collection unit can collect both facial expressions and voice tone in a balanced manner. Thus, by adjusting the collection means based on the current environment, more appropriate data collection becomes possible. Some or all of the above-described processing in the collection unit may be performed using AI, or may be performed without using AI. For example, the collection unit can input current environment data into an AI model, and the AI can propose the optimal collection means.

The emotion recognition support system comprises a collection unit that corrects the collected data by considering the subject's physical condition at the time of collection. The collection unit corrects the collected data by considering the subject's physical condition (fatigue, health status, etc.) at the time of collection. The correction is performed, for example, by adjusting or filtering the data, but is not limited to such examples. For example, if the subject is tired, the collection unit focuses on collecting changes in facial expressions and corrects changes in voice tone. If the subject is in good health, the collection unit can collect both facial expressions and voice tone in a balanced manner. If the subject is in poor physical condition, the collection unit can focus on collecting changes in voice tone and correct changes in facial expressions. Thus, by correcting the collected data considering the physical condition, more accurate data collection becomes possible. Some or all of the above-described processing in the collection unit may be performed using AI, or may be performed without using AI. For example, the collection unit can input physical condition data into an AI model, and the AI can perform the correction of the collected data.

The emotion recognition support system comprises a collection unit that estimates the user's emotions and filters the data to be collected based on the estimated emotions of the user. The collection unit estimates the user's emotions and filters the data to be collected based on the estimated emotions of the user. The filtering is performed, for example, by selecting data or removing noise, but is not limited to such examples. For example, if the user is feeling stressed, the collection unit removes noise and collects subtle changes in facial expressions. If the user is relaxed, the collection unit can remove background noise and collect changes in voice tone. If the user is excited, the collection unit can filter and collect both facial expressions and voice tone. Thus, by filtering data based on the user's emotions, more accurate emotion analysis becomes possible. Some or all of the above-described processing in the collection unit may be performed using AI, or may be performed without using AI. For example, the collection unit can input the user's emotional data into an AI model, and the AI can perform filtering.

The emotion recognition support system comprises a collection unit that enhances the relevance of collected data by considering the subject's geographic location information at the time of collection. The collection unit enhances the relevance of collected data by considering the subject's geographic location information at the time of collection. The enhancement of relevance is performed, for example, by selecting or filtering data, but is not limited to such examples. For example, if the subject is in a park, the collection unit removes natural sounds and collects facial expressions and voice tone. If the subject is in an office, the collection unit can remove background conversation sounds and collect facial expressions and voice tone. If the subject is at home, the collection unit can remove household noise and collect facial expressions and voice tone. Thus, by considering geographic location information, the relevance of collected data can be enhanced and more accurate data collection becomes possible. Some or all of the above-described processing in the collection unit may be performed using AI, or may be performed without using AI. For example, the collection unit can input geographic location information into an AI model, and the AI can perform filtering to enhance the relevance of collected data.

The emotion recognition support system comprises a collection unit that analyzes the subject's social media activity at the time of collection and supplements relevant emotional data. The collection unit analyzes the subject's social media activity at the time of collection and supplements relevant emotional data. The supplementation is performed, for example, by adding or correcting data, but is not limited to such examples. For example, the collection unit analyzes the content of the subject's social media posts and supplements changes in emotions. The collection unit can also refer to the activities of the subject's friends on social media to supplement emotional data. The collection unit can also supplement emotional data based on the subject's check-in information on social media. Thus, by analyzing social media activity, relevant emotional data can be supplemented and more accurate emotion analysis becomes possible. Some or all of the above-described processing in the collection unit may be performed using AI, or may be performed without using AI. For example, the collection unit can input social media activity data into an AI model, and the AI can perform supplementation of emotional data.

The emotion recognition support system comprises a collection unit that customizes the collection method by reflecting the subject's past feedback at the time of collection. The collection unit customizes the collection method by reflecting the subject's past feedback at the time of collection. The customization is performed, for example, by selecting the collection means or changing settings, but is not limited to such examples. For example, the collection unit adjusts the collection method based on feedback previously provided by the subject. The collection unit can also preferentially use collection methods that the subject has preferred in the past. The collection unit can also analyze the subject's past feedback and propose the optimal collection method. Thus, by reflecting past feedback, the collection method can be customized and more accurate data collection becomes possible. Some or all of the above-described processing in the collection unit may be performed using AI, or may be performed without using AI. For example, the collection unit can input past feedback data into an AI model, and the AI can perform customization of the collection method.

The emotion recognition support system comprises an analysis unit that estimates the user's emotions and adjusts the analysis algorithm based on the estimated emotions of the user. The analysis unit estimates the user's emotions and adjusts the analysis algorithm based on the estimated emotions of the user. The adjustment is performed, for example, by setting algorithm parameters or weighting, but is not limited to such examples. For example, if the user is feeling stressed, the analysis unit uses an algorithm that emphasizes subtle changes in emotions. If the user is relaxed, the analysis unit can use an algorithm that emphasizes the overall tone of emotions. If the user is excited, the analysis unit can use an algorithm that emphasizes rapid changes in emotions. Thus, by adjusting the analysis algorithm based on the user's emotions, more accurate emotion analysis becomes possible. Some or all of the above-described processing in the analysis unit may be performed using AI, or may be performed without using AI. For example, the analysis unit can input the user's emotional data into an AI model, and the AI can perform adjustment of the analysis algorithm.

The emotion recognition support system comprises an analysis unit that improves accuracy by comparing the subject's emotional patterns with past data during analysis. The analysis unit improves accuracy by comparing the subject's emotional patterns with past data during analysis. The improvement of accuracy is performed, for example, by improving data accuracy or algorithms, but is not limited to such examples. For example, the analysis unit analyzes by comparing the current emotional patterns with the subject's past emotional data. The analysis unit can also refer to the subject's past emotional patterns to more accurately identify current emotions. The analysis unit can also use the subject's past emotional data to improve the accuracy of the analysis algorithm. Thus, by comparing with past data, the accuracy of emotion analysis can be improved. Some or all of the above-described processing in the analysis unit may be performed using AI, or may be performed without using AI. For example, the analysis unit can input past emotional data into an AI model, and the AI can perform analysis for accuracy improvement.

The emotion recognition support system comprises an analysis unit that tracks fluctuations in the subject's emotions in real time during analysis and updates the analysis results. The analysis unit tracks fluctuations in the subject's emotions in real time during analysis and updates the analysis results. The tracking is performed, for example, by monitoring emotional fluctuations in real time and updating the analysis results each time a fluctuation occurs, but is not limited to such examples. For example, the analysis unit updates the analysis results in real time each time the subject's emotions fluctuate. The analysis unit can also track fluctuations in the subject's emotions in real time and provide the latest analysis results. The analysis unit can also immediately update the analysis results if the subject's emotions fluctuate rapidly. Thus, by tracking emotional fluctuations in real time, the latest analysis results can be provided. Some or all of the above-described processing in the analysis unit may be performed using AI, or may be performed without using AI. For example, the analysis unit can input real-time emotional data into an AI model, and the AI can perform updating of the analysis results.

The emotion recognition support system comprises an analysis unit that considers external factors affecting the subject's emotions during analysis. The analysis unit considers external factors (such as weather, time of day, etc.) affecting the subject's emotions during analysis. The consideration is performed, for example, by collecting data on external factors and reflecting them in emotion analysis, but is not limited to such examples. For example, the analysis unit analyzes by considering the impact of bad weather on the subject's emotions. The analysis unit can also analyze by considering emotional fluctuations due to time of day. The analysis unit can also analyze by considering external factors (such as noise level, influence of surrounding people, etc.). Thus, by considering external factors, more accurate emotion analysis becomes possible. Some or all of the above-described processing in the analysis unit may be performed using AI, or may be performed without using AI. For example, the analysis unit can input external factor data into an AI model and reflect it in emotion analysis.

The emotion recognition support system comprises an analysis unit that estimates the user's emotions and adjusts the display method of analysis results based on the estimated emotions of the user. The analysis unit estimates the user's emotions and adjusts the display method of analysis results based on the estimated emotions of the user. The adjustment is performed, for example, by selecting the display format or changing settings, but is not limited to such examples. For example, if the user is feeling stressed, the analysis unit provides a simple and highly visible display method. If the user is relaxed, the analysis unit can provide a display method that includes detailed information. If the user is excited, the analysis unit can provide a visually stimulating display method. Thus, by adjusting the display method based on the user's emotions, more appropriate information provision becomes possible. Some or all of the above-described processing in the analysis unit may be performed using AI, or may be performed without using AI. For example, the analysis unit can input the user's emotional data into an AI model, and the AI can perform adjustment of the display method.

The emotion recognition support system comprises an analysis unit that performs emotion analysis by considering the subject's geographic background during analysis. The analysis unit performs emotion analysis by considering the subject's geographic background during analysis. The consideration is performed, for example, by collecting data on geographic background and reflecting it in emotion analysis, but is not limited to such examples. For example, if the subject is in an urban area, the analysis unit analyzes by considering urban-specific stress factors. If the subject is in a natural environment, the analysis unit can analyze by considering relaxation effects. If the subject is in a specific region, the analysis unit can analyze by considering the culture and customs specific to that region. Thus, by considering geographic background, more accurate emotion analysis becomes possible. Some or all of the above-described processing in the analysis unit may be performed using AI, or may be performed without using AI. For example, the analysis unit can input geographic background data into an AI model and reflect it in emotion analysis.

The emotion recognition support system comprises an analysis unit that improves analysis accuracy by referring to relevant literature and research data on the subject during analysis. The analysis unit improves analysis accuracy by referring to relevant literature and research data on the subject during analysis. The reference is performed, for example, by collecting and analyzing relevant literature and research data, but is not limited to such examples. For example, the analysis unit analyzes by referring to the latest research data on the subject's emotions. The analysis unit can also adjust the analysis algorithm based on relevant literature on the subject's emotions. The analysis unit can also improve analysis accuracy by referring to past research data on the subject's emotions. Thus, by referring to relevant literature and research data, analysis accuracy can be improved. Some or all of the above-described processing in the analysis unit may be performed using AI, or may be performed without using AI. For example, the analysis unit can input relevant literature and research data into an AI model, and the AI can perform analysis accuracy improvement.

The emotion recognition support system comprises an analysis unit that analyzes emotions by considering the subject's social background during analysis. The analysis unit analyzes emotions by considering the subject's social background (such as culture, customs, etc.) during analysis. The consideration is performed, for example, by collecting data on social background and reflecting it in emotion analysis, but is not limited to such examples. For example, the analysis unit analyzes emotions by considering the subject's cultural background. The analysis unit can also analyze emotions by considering the subject's customs and lifestyle. The analysis unit can also analyze emotional fluctuations based on the subject's social background. Thus, by considering social background, more accurate emotion analysis becomes possible. Some or all of the above-described processing in the analysis unit may be performed using AI, or may be performed without using AI. For example, the analysis unit can input social background data into an AI model and reflect it in emotion analysis.

The emotion recognition support system comprises a provision unit that estimates the user's emotions and determines the priority of information to be provided based on the estimated emotions of the user. The provision unit estimates the user's emotions and determines the priority of information to be provided based on the estimated emotions of the user. The determination of priority is performed, for example, based on the importance or urgency of the information, but is not limited to such examples. For example, if the user is feeling stressed, the provision unit prioritizes providing information with a relaxing effect. If the user is relaxed, the provision unit can prioritize providing detailed information. If the user is excited, the provision unit can prioritize providing visually stimulating information. Thus, by determining the priority of information based on the user's emotions, more appropriate information provision becomes possible. Some or all of the above-described processing in the provision unit may be performed using AI, or may be performed without using AI. For example, the provision unit can input the user's emotional data into an AI model, and the AI can perform determination of information priority.

The emotion recognition support system comprises a provision unit that optimizes the information provision method by referring to the subject's past responses at the time of provision. The provision unit optimizes the information provision method by referring to the subject's past responses at the time of provision. The optimization is performed, for example, by selecting the information provision means or changing settings, but is not limited to such examples. For example, the provision unit preferentially uses information provision methods that the subject has preferred in the past. The provision unit can also propose the optimal information provision method based on the subject's past responses. The provision unit can also analyze the subject's past responses and optimize the information provision method. Thus, by referring to past responses, the information provision method can be optimized and more appropriate information provision becomes possible. Some or all of the above-described processing in the provision unit may be performed using AI, or may be performed without using AI. For example, the provision unit can input past response data into an AI model, and the AI can perform optimization of the information provision method.

The emotion recognition support system comprises a provision unit that adjusts information provision based on the subject's current situation at the time of provision. The provision unit adjusts information provision based on the subject's current situation (such as being active or on break) at the time of provision. The adjustment is performed, for example, by changing the timing or format of information provision, but is not limited to such examples. For example, if the subject is active, the provision unit provides concise and to-the-point information. If the subject is on break, the provision unit can provide detailed information. The provision unit can also propose the optimal information provision method based on the subject's current situation. Thus, by adjusting information provision based on the current situation, more appropriate information provision becomes possible. Some or all of the above-described processing in the provision unit may be performed using AI, or may be performed without using AI. For example, the provision unit can input current situation data into an AI model, and the AI can perform adjustment of information provision.

The emotion recognition support system comprises a provision unit that corrects information provision by considering the subject's physical condition at the time of provision. The provision unit corrects information provision by considering the subject's physical condition (such as fatigue, health status, etc.) at the time of provision. The correction is performed, for example, by changing the content or format of information, but is not limited to such examples. For example, if the subject is tired, the provision unit provides concise and to-the-point information. If the subject is in good health, the provision unit can provide detailed information. If the subject is in poor physical condition, the provision unit can provide information with a relaxing effect. Thus, by correcting information provision considering the physical condition, more appropriate information provision becomes possible. Some or all of the above-described processing in the provision unit may be performed using AI, or may be performed without using AI. For example, the provision unit can input physical condition data into an AI model, and the AI can perform correction of information provision.

The emotion recognition support system comprises a provision unit that estimates the user's emotions and adjusts the format of information to be provided based on the estimated emotions of the user. The provision unit estimates the user's emotions and adjusts the format of information to be provided based on the estimated emotions of the user. The adjustment is performed, for example, by changing the display format or notification method of information, but is not limited to such examples. For example, if the user is feeling stressed, the provision unit provides information in a visually simple format. If the user is relaxed, the provision unit can provide information in a format that includes detailed graphics or charts. If the user is excited, the provision unit can provide information in a visually stimulating format. Thus, by adjusting the format of information based on the user's emotions, more appropriate information provision becomes possible. Some or all of the above-described processing in the provision unit may be performed using AI, or may be performed without using AI. For example, the provision unit can input the user's emotional data into an AI model, and the AI can perform adjustment of the information format.

The emotion recognition support system comprises a provision unit that provides highly relevant information by considering the subject's geographic location information at the time of provision. The provision unit provides highly relevant information by considering the subject's geographic location information at the time of provision. The enhancement of relevance is performed, for example, by selecting or filtering information, but is not limited to such examples. For example, if the subject is in a specific region, the provision unit provides information related to that region. If the subject is traveling, the provision unit can provide information related to the travel destination. If the subject is at home, the provision unit can provide information about the area around the home. Thus, by considering geographic location information, highly relevant information provision becomes possible. Some or all of the above-described processing in the provision unit may be performed using AI, or may be performed without using AI. For example, the provision unit can input geographic location information into an AI model, and the AI can select highly relevant information.

The emotion recognition support system comprises a provision unit that analyzes the subject's social media activity at the time of provision and provides relevant information. The provision unit analyzes the subject's social media activity at the time of provision and provides relevant information. The analysis is performed, for example, by analyzing the content and reactions of social media posts, but is not limited to such examples. For example, the provision unit provides information about places where the subject has checked in on social media. The provision unit can also analyze the content of the subject's social media posts and provide relevant information. The provision unit can also refer to the activities of the subject's friends on social media to provide relevant information. Thus, by analyzing social media activity, relevant information provision becomes possible. Some or all of the above-described processing in the provision unit may be performed using AI, or may be performed without using AI. For example, the provision unit can input social media activity data into an AI model, and the AI can select relevant information.

The emotion recognition support system comprises a provision unit that customizes the information provision method by reflecting the subject's past feedback at the time of provision. The provision unit customizes the information provision method by reflecting the subject's past feedback at the time of provision. The customization is performed, for example, by selecting the information provision means or changing settings, but is not limited to such examples. For example, the provision unit preferentially uses information provision methods that the subject has preferred in the past. The provision unit can also propose the optimal information provision method based on the subject's past feedback. The provision unit can also analyze the subject's past feedback and customize the information provision method. Thus, by reflecting past feedback, the information provision method can be customized and more appropriate information provision becomes possible. Some or all of the above-described processing in the provision unit may be performed using AI, or may be performed without using AI. For example, the provision unit can input past feedback data into an AI model, and the AI can perform customization of the information provision method.

The emotion recognition support system comprises a learning unit that estimates the user's emotions and selects learning data based on the estimated emotions of the user. The learning unit estimates the user's emotions and selects learning data based on the estimated emotions of the user. The selection is performed, for example, by determining the type or priority of learning data, but is not limited to such examples. For example, if the user is feeling stressed, the learning unit prioritizes learning data related to stress reduction. If the user is relaxed, the learning unit can prioritize learning data with a relaxing effect. If the user is excited, the learning unit can prioritize learning data that calms excitement. Thus, by selecting learning data based on the user's emotions, more effective learning becomes possible. Some or all of the above-described processing in the learning unit may be performed using AI, or may be performed without using AI. For example, the learning unit can input the user's emotional data into an AI model, and the AI can perform selection of learning data.

The emotion recognition support system comprises a learning unit that optimizes the learning algorithm by referring to past learning data during learning. The learning unit optimizes the learning algorithm by referring to past learning data during learning. The optimization is performed, for example, by adjusting algorithms or setting parameters, but is not limited to such examples. For example, the learning unit adjusts the current learning algorithm based on past learning data. The learning unit can also refer to past learning data to find the optimal learning pattern. The learning unit can also use past learning data to improve the accuracy of the learning algorithm. Thus, by referring to past learning data, the learning algorithm can be optimized and the accuracy of learning can be improved. Some or all of the above-described processing in the learning unit may be performed using AI, or may be performed without using AI. For example, the learning unit can input past learning data into an AI model, and the AI can perform optimization of the learning algorithm.

The emotion recognition support system comprises a learning unit that analyzes fluctuations in the subject's emotional patterns during learning and adjusts the update frequency of learning data. The learning unit analyzes fluctuations in the subject's emotional patterns during learning and adjusts the update frequency of learning data. The adjustment is performed, for example, by setting the update frequency or weighting data, but is not limited to such examples. For example, if the subject's emotional patterns fluctuate frequently, the learning unit increases the update frequency of learning data. If the subject's emotional patterns are stable, the learning unit can decrease the update frequency of learning data. The learning unit can also analyze fluctuations in the subject's emotional patterns and set the optimal update frequency. Thus, by analyzing fluctuations in emotional patterns, the update frequency of learning data can be optimized and the accuracy of learning can be improved. Some or all of the above-described processing in the learning unit may be performed using AI, or may be performed without using AI. For example, the learning unit can input fluctuation data of emotional patterns into an AI model, and the AI can perform adjustment of the update frequency.

The emotion recognition support system comprises a learning unit that updates learning data by reflecting the subject's feedback during learning. The learning unit updates learning data by reflecting the subject's feedback during learning. The update is performed, for example, by collecting and analyzing feedback, but is not limited to such examples. For example, the learning unit updates learning data based on feedback provided by the subject. The learning unit can also analyze the subject's feedback and adjust the learning algorithm. The learning unit can also improve the accuracy of learning data by reflecting the subject's feedback. Thus, by reflecting feedback, learning data can be updated and the accuracy of learning can be improved. Some or all of the above-described processing in the learning unit may be performed using AI, or may be performed without using AI. For example, the learning unit can input feedback data into an AI model, and the AI can perform updating of learning data.

The emotion recognition support system comprises a learning unit that estimates the user's emotions and adjusts the frequency of learning based on the estimated emotions of the user. The learning unit estimates the user's emotions and adjusts the frequency of learning based on the estimated emotions of the user. The adjustment is performed, for example, by setting the timing or frequency of learning, but is not limited to such examples. For example, if the user is feeling stressed, the learning unit increases the frequency of learning. If the user is relaxed, the learning unit can decrease the frequency of learning. If the user is excited, the learning unit can adjust the frequency of learning. Thus, by adjusting the frequency of learning based on the user's emotions, more effective learning becomes possible. Some or all of the above-described processing in the learning unit may be performed using AI, or may be performed without using AI. For example, the learning unit can input the user's emotional data into an AI model, and the AI can perform adjustment of the learning frequency.

The emotion recognition support system comprises a learning unit that weights learning data based on the submission timing of the subject's emotional data during learning. The learning unit weights learning data based on the submission timing of the subject's emotional data during learning. The weighting is performed, for example, based on the importance or freshness of the data, but is not limited to such examples. For example, the learning unit weights learning data based on the timing when the subject submitted emotional data. The learning unit can also adjust the learning algorithm by considering the submission timing of the subject's emotional data. The learning unit can also set the priority of learning data based on the submission timing of the subject's emotional data. Thus, by weighting learning data based on the submission timing of emotional data, the accuracy of learning can be improved. Some or all of the above-described processing in the learning unit may be performed using AI, or may be performed without using AI. For example, the learning unit can input the submission timing of emotional data into an AI model, and the AI can perform weighting.

The emotion recognition support system comprises a learning unit that expands learning data by integrating information from different data sources during learning. The learning unit expands learning data by integrating information from different data sources during learning. The integration is performed, for example, by collecting and integrating data, but is not limited to such examples. For example, the learning unit integrates emotional data from different data sources for learning. The learning unit can also expand learning data based on information from different data sources. The learning unit can also use data from different data sources to improve the accuracy of the learning algorithm. Thus, by integrating information from different data sources, learning data can be expanded and the accuracy of learning can be improved. Some or all of the above-described processing in the learning unit may be performed using AI, or may be performed without using AI. For example, the learning unit can input information from different data sources into an AI model, and the AI can perform data integration.

The emotion recognition support system comprises a learning unit that adjusts the learning algorithm by considering the subject's social background during learning. The learning unit adjusts the learning algorithm by considering the subject's social background (such as culture, customs, etc.) during learning. The adjustment is performed, for example, by setting algorithm parameters or weighting, but is not limited to such examples. For example, the learning unit adjusts the learning algorithm by considering the subject's cultural background. The learning unit can also adjust the learning algorithm by considering the subject's customs and lifestyle. The learning unit can also optimize the learning algorithm based on the subject's social background. Thus, by considering social background, the learning algorithm can be optimized and the accuracy of learning can be improved. Some or all of the above-described processing in the learning unit may be performed using AI, or may be performed without using AI. For example, the learning unit can input social background data into an AI model, and the AI can perform adjustment of the algorithm.

Below is a brief explanation of the processing flow of Example 2 of the Embodiment.
Step 1: The collection unit collects the facial expressions and voice tone of the subject. For example, the collection unit uses a microphone-camera type device to collect facial expressions such as smiles, anger, sadness, and voice characteristics such as high pitch, low pitch, and intonation.
Step 2: The analysis unit analyzes the information collected by the collection unit and identifies the emotions of the subject. For example, the analysis unit uses an emotion recognition algorithm to analyze facial expression data and voice tone, and determines whether the subject is smiling, angry, etc.
Step 3: The provision unit provides the analysis results obtained by the analysis unit in real time. For example, the provision unit can provide the results to caregivers or child welfare workers through an earphone-type device, display them on a display, or notify them by voice.
Step 4: The learning unit performs self-learning to respond to the individual care needs of users based on the information provided by the provision unit. For example, the learning unit uses a machine learning algorithm to learn what kind of emotions a specific subject shows in specific situations and what kind of responses are preferred, and presents the optimal approach.

The specific processing unit 290 sends the results of specific processing to the smart device 14. In the smart device 14, the control unit 46A causes the output device 40 to output the results of specific processing. The microphone 38B acquires voice indicating user input in response to the results of specific processing. The control unit 46A sends the voice data indicating user input acquired by the microphone 38B to the data processing device 12. In the data processing device 12, the specific processing unit 290 acquires the voice data.

The data generation model 58 is a so-called generative AI (Artificial Intelligence). An example of the data generation model 58 is a generative AI such as ChatGPT (registered trademark) (Internet search <URL: https://openai.com/blog/chatgpt>). The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

Moreover, the processing by the data processing system 10 described above is executed by the specific processing unit 290 of the data processing device 12 or the control unit 46A of the smart device 14, but it may be executed by both the specific processing unit 290 of the data processing device 12 and the control unit 46A of the smart device 14. Additionally, the specific processing unit 290 of the data processing device 12 acquires or collects necessary information for processing from the smart device 14 or external devices, and the smart device 14 acquires or collects necessary information for processing from the data processing device 12 or external devices.

The correspondence between each unit and the devices or control units is not limited to the examples described above, and various modifications are possible.

### Second Embodiment

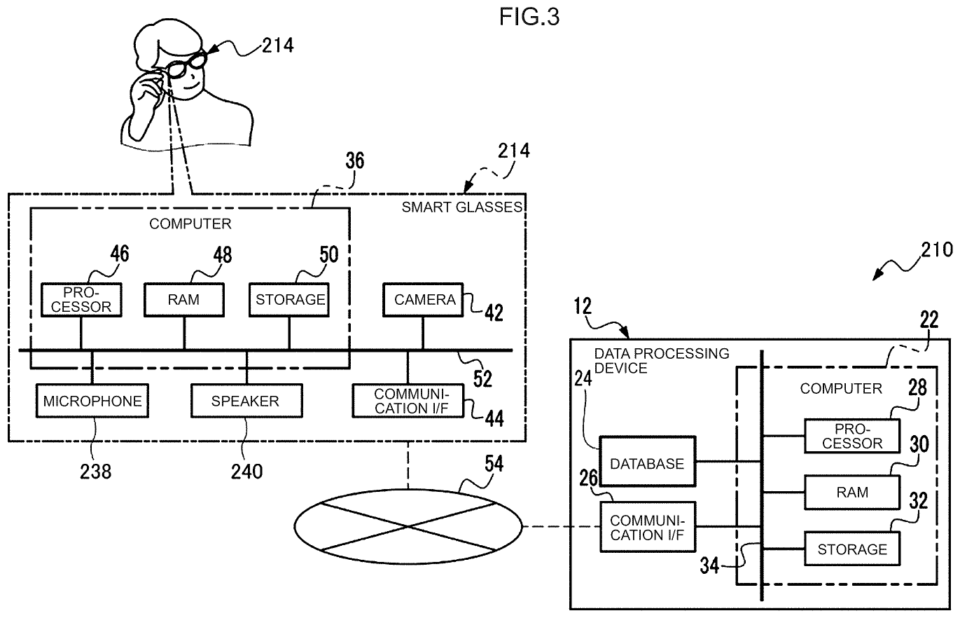

FIG. 3 shows an example configuration of a data processing system 210 according to the second embodiment.

As shown in FIG. 3, the data processing system 210 comprises a data processing device 12 and smart glasses 214. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The smart glasses 214 comprise a computer 36, a microphone 238, a speaker 240, a camera 42, and a communication I/F 44. The computer 36 comprises a processor 46, RAM 48, and storage 50. The processor 46, RAM 48, and storage 50 are connected to a bus 52. The microphone 238, speaker 240, and camera 42 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

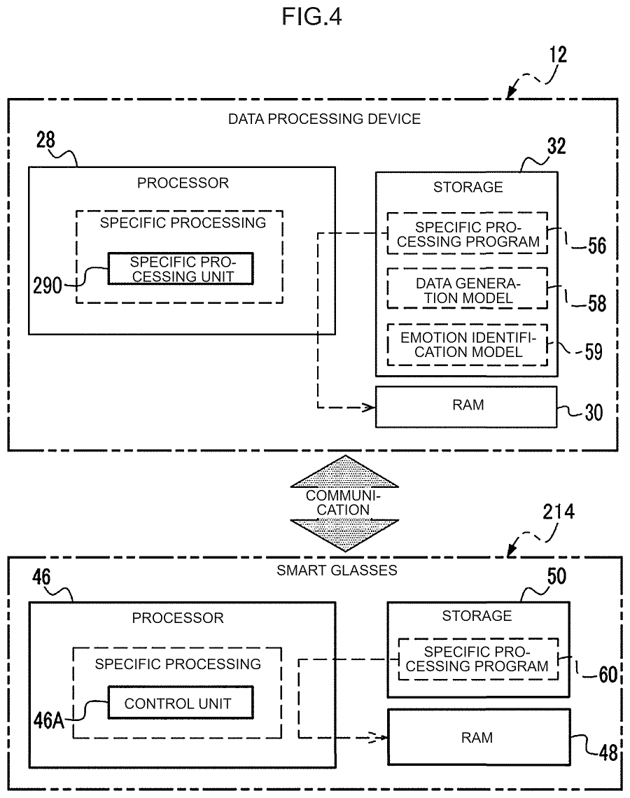

FIG. 4 shows an example of the main functions of the data processing device 12 and smart glasses 214. As shown in FIG. 4, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the smart glasses 214, specific processing is performed by the processor 46. The storage 50 stores a specific processing program 60. The processor 46 reads the specific processing program 60 from the storage 50 and executes it on the RAM 48. The specific processing is realized by the processor 46 operating as a control unit 46A according to the specific processing program 60 executed on the RAM 48. The smart glasses 214 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the smart glasses 214. In the smart glasses 214, the control unit 46A causes the speaker 240 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 210 according to the second embodiment performs the same processing as the data processing system 10 according to the first embodiment. The processing by the data processing system 210 is executed by the specific processing unit 290 of the data processing device 12 or the control unit 46A of the smart glasses 214, but it may be executed by both the specific processing unit 290 of the data processing device 12 and the control unit 46A of the smart glasses 214. Additionally, the specific processing unit 290 of the data processing device 12 acquires or collects necessary information for processing from the smart glasses 214 or external devices, and the smart glasses 214 acquires or collects necessary information for processing from the data processing device 12 or external devices.

The correspondence between each unit and the devices or control units is not limited to the examples described above, and various modifications are possible.

### Third Embodiment

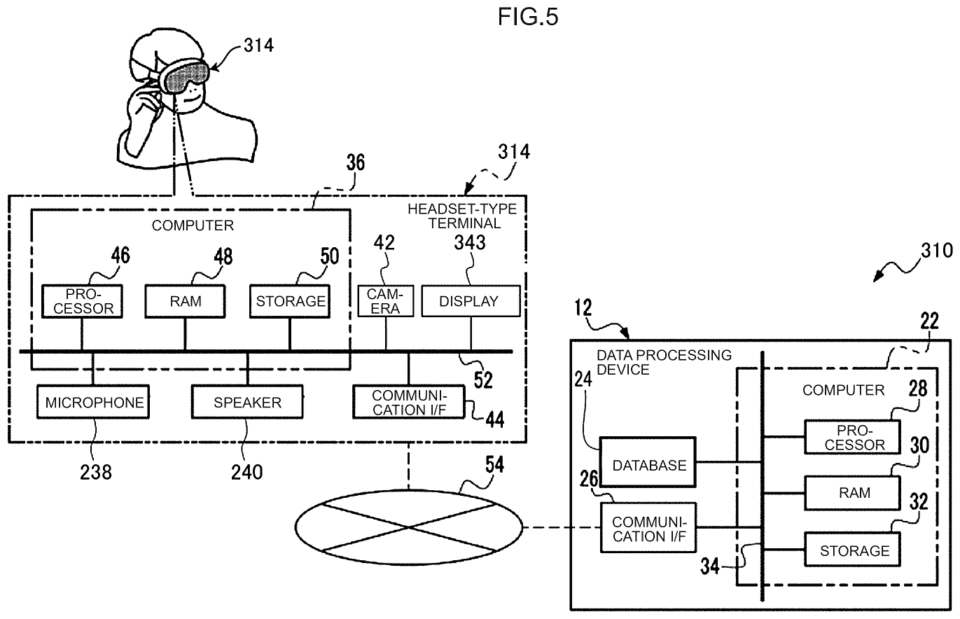

FIG. 5 shows an example configuration of a data processing system 310 according to the third embodiment.

As shown in FIG. 5, the data processing system 310 comprises a data processing device 12 and a headset-type terminal 314. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The headset-type terminal 314 comprises a computer 36, a microphone 238, a speaker 240, a camera 42, a communication I/F 44, and a display 343. The computer 36 comprises a processor 46, RAM 48, and storage 50. The processor 46, RAM 48, and storage 50 are connected to a bus 52. The microphone 238, speaker 240, camera 42, and display 343 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

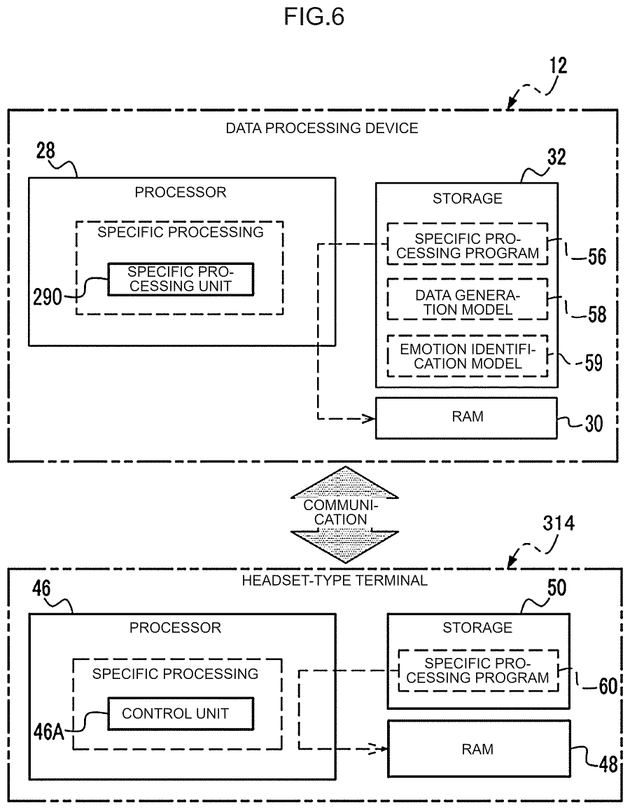

FIG. 6 shows an example of the main functions of the data processing device 12 and the headset-type terminal 314. As shown in FIG. 6, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the headset-type terminal 314, specific processing is performed by the processor 46. The storage 50 stores a specific program 60. The processor 46 reads the specific program 60 from the storage 50 and executes it on the RAM 48. The specific processing is realized by the processor 46 operating as a control unit 46A according to the specific program 60 executed on the RAM 48. The headset-type terminal 314 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the headset-type terminal 314. In the headset-type terminal 314, the control unit 46A causes the speaker 240 and the display 343 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 310 according to the third embodiment performs the same processing as the data processing system 10 according to the first embodiment. The processing by the data processing system 310 is executed by the specific processing unit 290 of the data processing device 12 or the control unit 46A of the headset-type terminal 314, but it may be executed by both the specific processing unit 290 of the data processing device 12 and the control unit 46A of the headset-type terminal 314. Additionally, the specific processing unit 290 of the data processing device 12 acquires or collects necessary information for processing from the headset-type terminal 314 or external devices, and the headset-type terminal 314 acquires or collects necessary information for processing from the data processing device 12 or external devices.

The correspondence between each unit and the devices or control units is not limited to the examples described above, and various modifications are possible.

### Fourth Embodiment

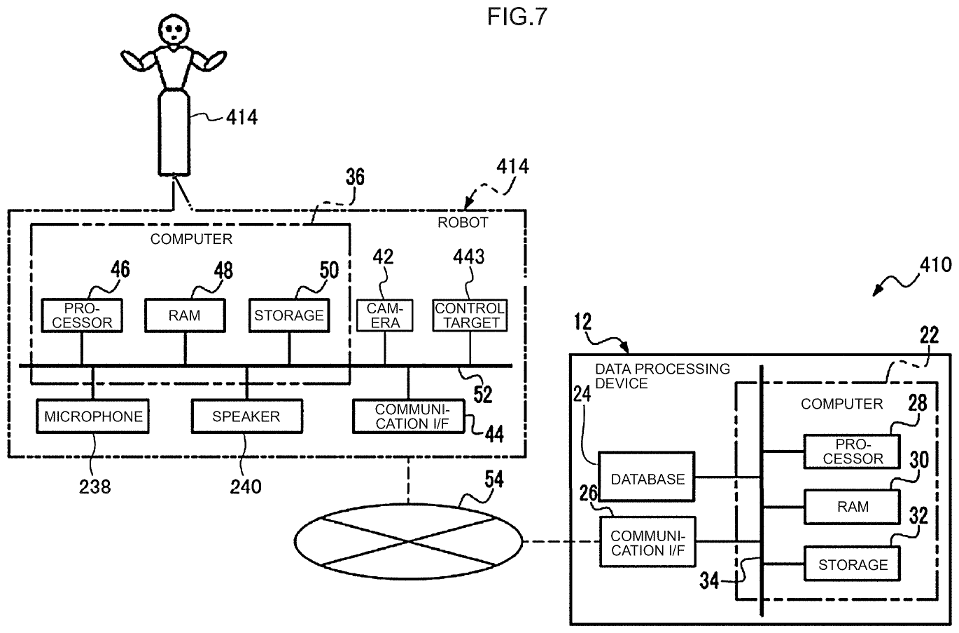

FIG. 7 shows an example configuration of a data processing system 410 according to the fourth embodiment.

As shown in FIG. 7, the data processing system 410 comprises a data processing device 12 and a robot 414. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The robot 414 comprises a computer 36, a microphone 238, a speaker 240, a camera 42, a communication I/F 44, and a control target 443. The computer 36 comprises a processor 46, RAM 48, and storage 50. The processor 46, RAM 48, and storage 50 are connected to a bus 52. The microphone 238, speaker 240, camera 42, and control target 443 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS image sensors or CCD image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

The control target 443 includes a display device, LEDS for the eyes, and motors for driving arms, hands, and feet, among others. The posture and gestures of the robot 414 are controlled by controlling the motors for the arms, hands, and feet, among others. Some emotions of the robot 414 can be expressed by controlling these motors. Additionally, the expression of the robot 414 can be expressed by controlling the lighting state of the LEDs for the eyes of the robot 414.

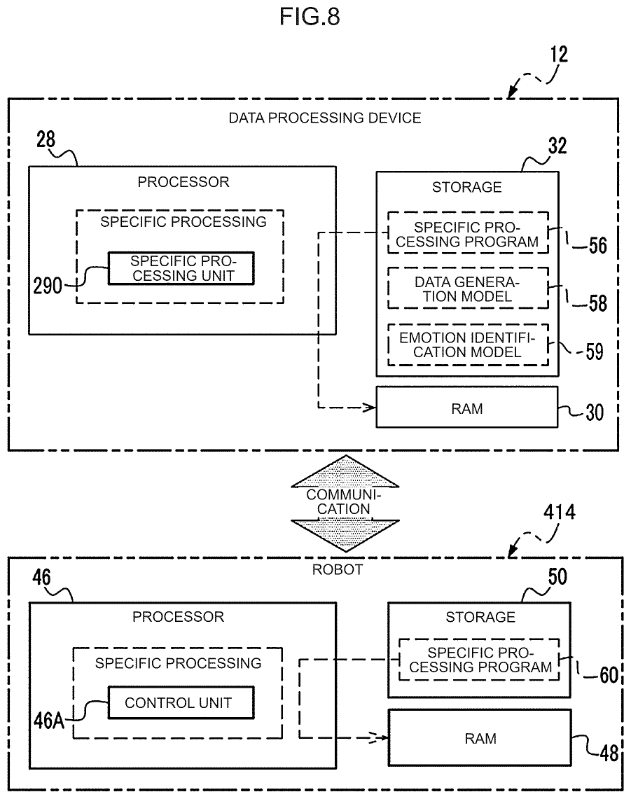

FIG. 8 shows an example of the main functions of the data processing device 12 and the robot 414. As shown in FIG. 8, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the robot 414, specific processing is performed by the processor 46. The storage 50 stores a specific program 60. The processor 46 reads the specific program 60 from the storage 50 and executes it on the RAM 48. The specific processing is realized by the processor 46 operating as a control unit 46A according to the specific program 60 executed on the RAM 48. The robot 414 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the robot 414. In the robot 414, the control unit 46A causes the speaker 240 and the control target 443 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 410 according to the fourth embodiment performs the same processing as the data processing system 10 according to the first embodiment. The processing by the data processing system 410 is executed by the specific processing unit 290 of the data processing device 12 or the control unit 46A of the robot 414, but it may be executed by both the specific processing unit 290 of the data processing device 12 and the control unit 46A of the robot 414. Additionally, the specific processing unit 290 of the data processing device 12 acquires or collects necessary information for processing from the robot 414 or external devices, and the robot 414 acquires or collects necessary information for processing from the data processing device 12 or external devices.

The correspondence between each unit and the devices or control units is not limited to the examples described above, and various modifications are possible.

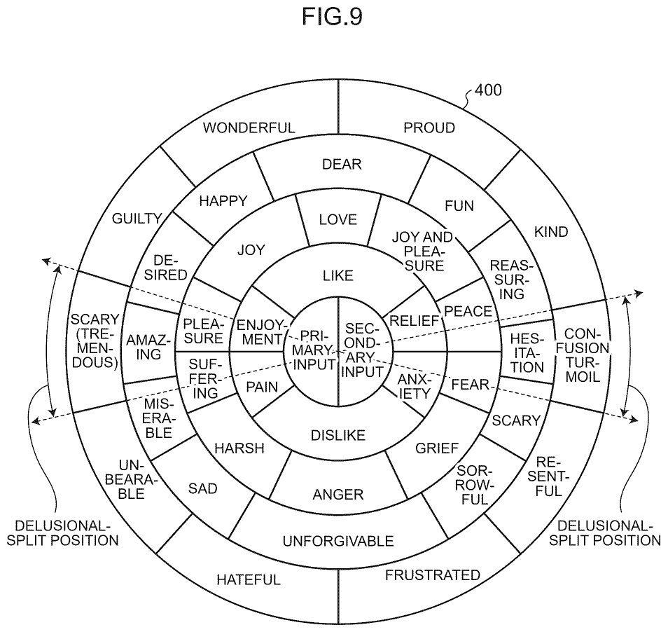

Note that the emotion identification model 59 as an emotion engine may determine the user's emotions according to a specific mapping. Specifically, the emotion identification model 59 may determine the user's emotions according to an emotion map, which is a specific mapping (see FIG. 9). Similarly, the emotion identification model 59 may determine the robot's emotions, and the specific processing unit 290 may perform specific processing using the robot's emotions.

FIG. 9 is a diagram showing an emotion map 400 where multiple emotions are mapped. In the emotion map 400, emotions are arranged concentrically radiating from the center. The closer to the center of the concentric circles, the more primitive the state of emotions is arranged. On the outer side of the concentric circles, emotions representing states and behaviors arising from mood are arranged. Emotions encompass concepts including emotional and mental states. On the left side of the concentric circles, emotions generally generated from reactions occurring in the brain are arranged. On the right side of the concentric circles, emotions generally induced by situational judgment are arranged. On the top and bottom of the concentric circles, emotions generated from reactions occurring in the brain and induced by situational judgment are arranged. Additionally, on the upper side of the concentric circles, “pleasant” emotions are arranged, and on the lower side, “unpleasant” emotions are arranged. In this way, in the emotion map 400, multiple emotions are mapped based on the structure from which emotions arise, and emotions that tend to occur simultaneously are mapped nearby.

These emotions are distributed in the 3 o'clock direction of the emotion map 400, and they usually move back and forth around reassurance and anxiety. In the right half of the emotion map 400, situational recognition takes precedence over internal sensations, giving a calm impression.

The inner side of the emotion map 400 represents the mind, and the outer side represents behavior, so the further out on the emotion map 400, the more visible (expressed in behavior) emotions become.

Here, human emotions are based on various balances like posture and blood sugar levels, and when these balances move away from the ideal, they indicate discomfort, and when they approach the ideal, they indicate comfort. In robots, cars, motorcycles, etc., emotions can be created based on various balances like posture and battery level, indicating discomfort when these balances move away from the ideal and comfort when they approach the ideal. The emotion map may be generated based on Dr. Mitsuyoshi's emotion map (Research on speech emotion recognition and brain physiological signal analysis systems related to emotions, Tokushima University, Doctoral dissertation: https://ci.nii.ac.jp/naid/500000375379). In the left half of the emotion map, emotions belonging to the domain called “reactions,” where sensations take precedence, are aligned. Additionally, in the right half of the emotion map, emotions belonging to the domain called “situations,” where situational recognition takes precedence, are aligned.

In the emotion map, two emotions that promote learning are defined. One is a negative emotion around “repentance” or “reflection” on the situation side. In other words, when a negative emotion arises in the robot, like “I never want to feel this way again” or “I don't want to be scolded again.” The other is an emotion around “desire” on the reaction side, which is positive. In other words, it is a positive feeling like “I want more” or “I want to know more.”

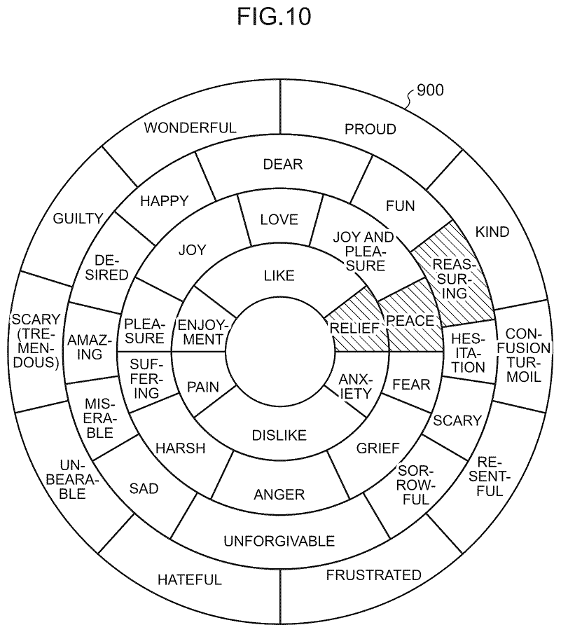

The emotion identification model 59 inputs user input into a pre-learned neural network, acquires emotion values indicating each emotion shown in the emotion map 400, and determines the user's emotions. This neural network is pre-learned based on multiple training data consisting of user input and combinations of emotion values indicating each emotion shown in the emotion map 400. Additionally, this neural network is learned so that emotions placed near each other in the emotion map 900 shown in FIG. 10 have similar values. FIG. 10 shows an example where multiple emotions like “reassured,” “calm,” and “confident” have similar emotion values.

In the above embodiments, an example form where specific processing is performed by a single computer 22 was described, but the technology disclosed herein is not limited to this, and distributed processing for specific processing by multiple computers including the computer 22 may be performed.

In the above embodiments, an example form where the specific processing program 56 is stored in the storage 32 was described, but the technology disclosed herein is not limited to this. For example, the specific processing program 56 may be stored in portable non-transitory storage media readable by a computer, such as a USB (Universal Serial Bus) memory. The specific processing program 56 stored in non-transitory storage media is installed in the computer 22 of the data processing device 12. The processor 28 executes specific processing according to the specific processing program 56.

Additionally, the specific processing program 56 may be stored in a storage device, such as a server connected to the data processing device 12 via the network 54, and downloaded and installed on the computer 22 in response to requests from the data processing device 12.

Furthermore, it is not necessary to store all of the specific processing program 56 in storage devices such as servers connected to the data processing device 12 via the network 54 or all in the storage 32, and a part of the specific processing program 56 may be stored.

Various processors, as shown next, can be used as hardware resources for executing specific processing. As processors, general-purpose processors that function as hardware resources for executing specific processing by executing software, i.e., programs, such as a CPU, can be mentioned. Additionally, as processors, dedicated electrical circuits with circuit configurations specially designed to execute specific processing, such as FPGA (Field-Programmable Gate Array), PLD (Programmable Logic Device), or ASIC (Application Specific Integrated Circuit), can be mentioned. Each processor has a built-in or connected memory, and each processor executes specific processing using the memory.

Hardware resources for executing specific processing may be composed of one of these various processors or a combination of two or more processors of the same or different types (e.g., a combination of multiple FPGAs or a combination of a CPU and FPGA). Additionally, hardware resources for executing specific processing may be a single processor.

As an example of composing with a single processor, firstly, there is a form where one or more CPUs and software are combined to constitute a single processor, which functions as hardware resources for executing specific processing. Secondly, there is a form using a processor, such as SoC (System-on-a-chip), that realizes the function of an entire system including multiple hardware resources for executing specific processing with a single IC chip. In this way, specific processing is realized using one or more of the various processors as hardware resources.

Furthermore, as a hardware structure of these various processors, more specifically, electrical circuits combined with circuit elements such as semiconductor elements can be used. Additionally, the specific processing described above is merely one example. Therefore, it goes without saying that unnecessary steps may be deleted, new steps may be added, or the order of processing may be changed within the scope not departing from the gist.

Additionally, in the examples described above, the explanation was divided into the first embodiment to the fourth embodiment, but parts or all of these embodiments may be combined. Additionally, the smart device 14, smart glasses 214, headset-type terminal 314, and robot 414 are examples, and each may be combined, or other devices may be used. Additionally, the examples described above were explained by dividing into form example 1 and form example 2, but these may be combined.

The descriptions and drawings shown above are detailed explanations of parts related to the technology disclosed herein and are merely examples of the technology disclosed herein. For example, the explanations regarding configurations, functions, actions, and effects above are explanations regarding examples of configurations, functions, actions, and effects of parts related to the technology disclosed herein. Therefore, it goes without saying that within the scope not departing from the gist of the technology disclosed herein, unnecessary parts may be deleted, new elements may be added, or replacements may be made to the descriptions and drawings shown above. Additionally, to avoid complexity and facilitate understanding of parts related to the technology disclosed herein, explanations concerning technical common knowledge and the like that do not require special explanation for enabling the implementation of the technology disclosed herein are omitted in the descriptions and drawings shown above.

All documents, patent applications, and technical standards described in this specification are incorporated by reference to the same extent as if each document, patent application, and technical standard were specifically and individually stated to be incorporated by reference in this specification.

What is claimed is:

1. A system comprising: a collection unit that collects the facial expressions and voice tone of a subject; an analysis unit that analyzes the information collected by the collection unit and identifies the emotions of the subject; a provision unit that provides the analysis results obtained by the analysis unit in real time; and a learning unit that performs self-learning to respond to the individual care needs of users based on the information provided by the provision unit.

2. The system according to claim 1, wherein the collection unit collects the facial expressions or voice tone of the subject using a microphone-camera type device.

3. The system according to claim 1, wherein the analysis unit analyzes the collected information and identifies the emotions of the subject.

4. The system according to claim 1, wherein the provision unit provides the analysis results in real time through an earphone-type device.

5. The system according to claim 1, wherein the learning unit learns the emotional patterns of a specific subject and presents an optimal approach.

6. The system according to claim 1, wherein the provision unit aims to reduce the burden on caregivers and child welfare workers and to improve the quality of welfare services.

7. The system according to claim 1, wherein the collection unit estimates the user's emotions and determines the priority of collecting facial expressions or voice tone based on the estimated emotions of the user.

8. The system according to claim 1, wherein the collection unit optimizes the collection method by referring to the subject's past emotional data at the time of collection.
